(19) United States
(12) Patent Application Publication
(10) Pub. No.: US 2023/0184639 A1
(43) Pub. Date: Jun. 15, 2023

(54) INFORMATION PROCESSING DEVICE, INFORMATION PROCESSING METHOD, COMPUTER PROGRAM, AND TARGET MOLECULE DETECTION SYSTEM

(71) Applicant: SONY GROUP CORPORATION, TOKYO (JP)

(72) Inventor: TOMOHIKO NAKAMURA, TOKYO (JP)

(21) Appl. No.: 17/925,867

(22) PCT Filed: Apr. 16, 2021
(86) PCT No.: PCT/JP2021/015679

§ 371 Date: Nov. 17, 2022

(30) Foreign Application Priority Data
May 29, 2020 (JP) 2020-094723

(51) Int. Cl. G01N 1/30; G01N 21/64; G01N 33/483

(52) U.S. Cl. CPC G01N 1/30; G01N 21/64; G01N 33/483

(57) ABSTRACT

To provide a technique for supporting setting of staining conditions for a reagent containing binding molecules when target molecules are detected and/or analyzed.

There is provided an information processing device including a signal acquisition unit that acquires a signal derived from a sample including a biological sample, a processing unit that calculates immunostaining conditions of a reagent for the sample including the biological sample based on the signal, and an output unit that outputs the immunostaining conditions, wherein the signal includes a signal derived from the reagent containing target molecules in the biological sample and binding molecules that are able to bind to the target molecules and/or a signal derived from non-target molecules and the reagent.

### TECHNICAL FIELD

The present technology relates to an information processing device, an information processing method, a computer program, and a target molecule detection system.

### BACKGROUND ART

Various analyses using labels have been performed to analyze various molecules. For example, molecules such as antigen proteins are detected and/or analyzed using antibodies labeled with a plurality of fluorescent dyes using a flow cytometer or a microscope. In addition, in addition to the antigen-antibody reaction, detection and analysis of molecules according to nucleic acid hybridization using a fluorescence-labeled nucleic acid probe and detection and analysis of enzyme molecules using fluorescence-labeled substrates are widely performed. Various fluorescent dyes are used in these detections and/or analyses. Each fluorescent dye has unique properties, for example, a unique fluorescence spectrum and fluorescence intensity.

For example, PTL 1 below describes an invention regarding a technique for analyzing the type of fluorescence emitted from microparticles (paragraph 0001). PTL 1 below describes “a data display method of displaying a fluorescence spectrum obtained by integrating or averaging detection data obtained by simultaneously detecting fluorescence emitted from microparticles flowing through a channel in a plurality of wavelength ranges for a plurality of microparticles” (claim 1).

### CITATION LIST

### Patent Literature

[PTL 1]
JP 2014-206551 A

### SUMMARY

### Technical Problem

When target molecules such as antigens and nucleic acids are stained with a reagent containing binding molecules such as antibodies and nucleic acid probes and the target molecules are then detected and/or analyzed, the reactivity influences detection accuracy. In addition, binding molecules bind and/or adsorb to molecules other than the target molecules, and thus the detection accuracy may decrease.

In addition, in order to detect and/or analyze target molecules, setting of staining conditions using a reagent containing binding molecules is directly performed by a user who performs detection and/or analysis, but even for the experienced user, it is a time-consuming task. In addition, it cannot be denied that the set staining conditions may not be suitable for detection and/or analysis of target molecules.

Therefore, a main object of the present technology is to provide a technique for supporting setting of staining conditions for a reagent containing binding molecules when target molecules are detected and/or analyzed.

### Solution to Problem

The present technology provides an information processing device including a signal acquisition unit that acquires a signal derived from a sample including a biological sample;

a processing unit that calculates immunostaining conditions of a reagent for the sample based on the signal; and

an output unit that outputs the immunostaining conditions,

wherein the signal includes a signal derived from the reagent containing target molecules in the biological sample and binding molecules that are able to bind to the target molecules and/or a signal derived from non-target molecules and the reagent.

The signal may include at least one of a signal, a specific signal/background, and a specific signal/non-specific signal.

The processing unit may calculate immunostaining conditions of the reagent for the sample including the biological sample based on signals derived from the sample stained with a plurality of reagent concentrations and a threshold value.

The threshold value may be the maximum signal among the signals derived from the sample stained with a plurality of reagent concentrations.

The processing unit may calculate immunostaining conditions of the reagent for the sample including the biological sample based on the signal derived from the sample stained with at least one reagent concentration and reagent information referred to in a database.

The processing unit may calculate immunostaining conditions of the reagent for the sample including the biological sample based on the signals derived from the sample stained with a plurality of reagent concentrations and a threshold value extracted from region information.

The processing unit may determine a region based on the signal and/or bright field image.

The region may include morphology information of the biological sample.

In this case, the morphology information may include a cell membrane and a nuclear distribution, or a cell morphology obtained by segmentation.

The processing unit may compare a plurality of determined regions.

The processing unit may compare a plurality of regions including at least one of a cell membrane, a cell nucleus, a specific binding region and a non-specific binding region and analyze localization of the regions.

When the plurality of determined regions are single cells, the processing unit may exclude signals derived from single cells that overlap and/or are adjacent to each other within a predetermined distance.

The signal acquisition unit may acquire a fluorescence signal after autofluorescence separation and/or inter-dye color separation.

The output unit may output, as the immunostaining conditions, at least one of an antibody clone, an antibody concentration, an antigen-antibody reaction time, a reaction temperature, antigen activation conditions, a composition of a reaction solution, and stirring conditions.

The information processing device according to the present technology may further include a presentation unit that presents support information for staining conditions to a user based on the output immunostaining conditions.

In addition, the present technology provides an information processing method, including

a signal acquisition step in which a signal derived from a sample including a biological sample is acquired;

a processing step in which immunostaining conditions of a reagent for the sample are calculated based on the signal; and

an output step in which the immunostaining conditions are output,

wherein the signal includes a signal derived from a reagent containing target molecules in the biological sample and binding molecules that are able to bind to the target molecules and/or a signal derived from non-target molecules and the reagent.

In addition, the present technology provides a computer program causing a computer to implement:

a signal acquisition function of acquiring a signal derived from a sample including a biological sample;

a processing function of calculating immunostaining conditions of a reagent for the sample based on the signal; and

an output function of outputting the immunostaining conditions,

wherein the signal includes a signal derived from a reagent containing target molecules in the biological sample and binding molecules that are able to bind to the target molecules and/or a signal derived from non-target molecules and the reagent.

In addition, the present technology provides a target molecule detection system, including:

a signal acquisition unit that acquires a signal derived from a sample including a biological sample;

a processing unit that calculates immunostaining conditions of a reagent for the sample based on the signal;

an output unit that outputs the immunostaining conditions; and

a detection unit that detects a signal derived from the stained sample based on the output immunostaining conditions,

wherein the signal includes a signal derived from a reagent containing target molecules in the biological sample and binding molecules that are able to bind to the target molecules and/or a signal derived from non-target molecules and the reagent.

The target molecule detection system according to the present technology may further include a staining unit that stains the sample using the reagent.

The target molecule detection system according to the present technology may further include an analysis unit that analyzes the sample based on the signal detected by the detection unit.

### BRIEF DESCRIPTION OF DRAWINGS

FIG. 1A is a graph showing the relationship between the binding molecule concentration and the light intensity, instead of drawings. FIG. 1B is a graph showing the relationship between the binding molecule concentration and a specific signal/non-specific signal ratio, instead of drawings.

FIG. 2 is a block diagram showing an example of an information processing device 1 according to the present technology.

FIG. 3A is a graph showing the relationship between the binding molecule concentration and the light intensity, instead of drawings. FIG. 3B and FIG. 3B′ are graphs showing the relationship between the binding molecule concentration and a specific signal/non-specific signal ratio, instead of drawings.

FIG. 4 is a conceptual diagram showing an example of an information processing system 2 according to the present technology.

FIG. 5 is a block diagram showing an example of a target molecule detection device 3 according to the present technology.

FIG. 6 is a conceptual diagram showing an example of a target molecule detection system 4 according to the present technology.

FIG. 7 is a conceptual diagram showing an example of the target molecule detection system 4 according to the present technology different from FIG. 6.

FIG. 8 is a conceptual diagram showing an example of the target molecule detection system 4 according to the present technology different from FIG. 6 and FIG. 7.

FIG. 9 is a flowchart showing an example of an information processing method according to the present technology.

FIG. 10 is a flowchart showing an example of the information processing method according to the present technology different from FIG. 9.

FIG. 11 is a flowchart showing a specific example of immunostaining condition calculation S5 when region/morphology determination S2 is performed in the information processing method according to the present technology.

FIG. 12 is a flowchart showing a specific example of a case in which, in the information processing method according to the present technology, immunostaining condition calculation S5 is performed using information accumulated in a database.

FIG. 13 is a flowchart showing a first embodiment of a method of presenting immunostaining conditions for anti-PgR antibodies using the information processing device according to the present technology.

FIG. 14 is a flowchart showing the first embodiment of the method of presenting immunostaining conditions for anti-PgR antibodies using the information processing device according to the present technology.

FIG. 15 is a flowchart showing the first embodiment of the method of presenting immunostaining conditions for anti-PgR antibodies using the information processing device according to the present technology.

FIG. 16 is a flowchart showing the first embodiment of the method of presenting immunostaining conditions for anti-PgR antibodies using the information processing device according to the present technology.

FIG. 17 is a flowchart showing a second embodiment of the method of presenting immunostaining conditions for anti-PgR antibodies using the information processing device according to the present technology.

FIG. 18 is a flowchart showing a third embodiment of the method of presenting immunostaining conditions for anti-PgR antibodies using the information processing device according to the present technology.

FIG. 19 is a flowchart showing a fourth embodiment of the method of presenting immunostaining conditions for anti-PgR antibodies using the information processing device according to the present technology.

FIG. 20 is a flowchart showing a fifth embodiment-1 of the method of presenting immunostaining conditions for anti-PgR antibodies using the information processing device according to the present technology.

FIG. 21 is a flowchart showing a fifth embodiment-2 of the method of presenting immunostaining conditions for anti-PgR antibodies using the information processing device according to the present technology.

FIG. 22 is a flowchart showing a fifth embodiment-3 of the method of presenting immunostaining conditions for anti-PgR antibodies using the information processing device according to the present technology.

FIG. 23 is a flowchart showing the fifth embodiment-3 of the method of presenting immunostaining conditions for anti-PgR antibodies using the information processing device according to the present technology.

FIG. 24 is a flowchart showing the fifth embodiment-3 of the method of presenting immunostaining conditions for anti-PgR antibodies using the information processing device according to the present technology.

FIG. 25 is a flowchart showing the fifth embodiment-3 of the method of presenting immunostaining conditions for anti-PgR antibodies using the information processing device according to the present technology.

FIG. 26 is a flowchart showing a sixth embodiment of the method of presenting immunostaining conditions for anti-PgR antibodies using the information processing device according to the present technology.

FIG. 27 is a flowchart showing the sixth embodiment of the method of presenting immunostaining conditions for anti-PgR antibodies using the information processing device according to the present technology.

FIG. 28 is a flowchart showing a seventh embodiment of the method of presenting immunostaining conditions for anti-PgR antibodies using the information processing device according to the present technology.

### DESCRIPTION OF EMBODIMENTS

Hereinafter, preferable embodiments for implementing the present technology will be described with reference to the drawings. The embodiments described below show examples of representative embodiments of the present technology, but the scope of the present technology should not be narrowly understood based on the embodiments. Here, description will proceed in the following order.

1. Main Object and Basic Concept of Present Technology

2. Information Processing Device 1

(1) Target Molecules

(2) Binding Molecules

(3) Signal Acquisition Unit 11

(4) Processing Unit 12

(5) Output Unit 13

(6) Presentation Unit 14

(7) Storage Unit 15

(8) Display Unit 16

(9) User Interface 17

3. Information Processing System 2

4. Target Molecule Detection Device 3 and Target Molecule Detection System 4

(1) Detection Unit 31 and Detection Device 41

(2) Staining Unit 32 and Staining Device 42

(3) Analysis Unit 33 and Analysis Device 43

5. Computer Program

6. Information Processing Method

(1) Image Data Acquisition S1

(2) Region/Morphology Determination S2

(3) Conjugate Signal Determination S3

(4) Comparison of Conjugate Signal and Region/Morphology S4

(5) Immunostaining Condition Calculation S5

(6) Immunostaining Condition Presenting S6

[First Embodiment]

[Second Embodiment]

[Third Embodiment]

[Fourth Embodiment]

[Fifth Embodiment]

[Sixth Embodiment]

[Seventh Embodiment]

1. Main Object and Basic Concept of Present Technology

Generally, for example, an antigen-antibody reaction is represented by the following Formula 1.

$$[ Ab ] + [ Ag ] ⇄ [ Ab \cdot Ag complex ] \qquad [ Math . 1 ]$$

$${K}_{D} = [ Ab ] [ Ag ] / [ Ab - Ag complex ] = [ ( {[ Ab ]}_{0} - X ) \times ( {[ Ag ]}_{0} - X ) ] / X$$

$$X = [ Ab - Ag complex ] = [ ( {[ Ab ]}_{0} + {[ Ag ]}_{0} + {K}_{D} ) \pm √ {( {[ Ab ]}_{0} + {[ Ag ]}_{0} + {K}_{D} )}^{2} - 4 \times {{[ Ag ]}_{0} [ Ab ]}_{0} ) ] / 2$$

However, in practice, as shown in the following Formula 2, not only a specific reaction but also a non-specific reaction occur.

[Ab]+[Ag′]⇄[Ab−Ag′ complex]K_{D}′

[Ab]+[Ag″]⇄[Ab−Ag″ complex]K_{D}″

[Ab]+[Ag′″]⇄[Ab−Ag′″ complex]K_{D}′″

[Ab]+[Ag″″]⇄[Ab−Ag″″ complex]K_{D}″″ [Math. 2]

Thus, for example, in the antigen-antibody reaction, it is known that the reaction efficiency largely depends on the reaction conditions. That is, efficient search for appropriate conditions in which a strong specific reaction and a weak non-specific reaction are exhibited requires labor and technique.

Specifically, for example, when evaluation with a signal is performed, if the antibody concentration is too high, not only a specific signal but all signals become high, including a non-specific signal. Therefore, under optimal antibody concentration conditions with a lowered antibody concentration, the specific signal exhibits a high value and the non-specific signal becomes low. However, if the antibody concentration is further lowered, the specific signal is also lowered.

In addition, for example, when evaluation with a signal/background ratio is performed, if the antibody concentration is higher, all signals become high, and not only a specific signal but also a non-specific signal becomes high, and thus the background becomes high and the signal/background ratio is lowered. Therefore, under optimal antibody concentration conditions with a lowered antibody concentration, the specific signal exhibits a high value and the non-specific signal becomes low so that the signal/background ratio becomes high. However, if the antibody concentration is further lowered, all signals including a specific signal become low, and the signal/background ratio is lowered. Alternatively, if the antibody concentration is lowered, since the non-specific signal is close to 0 in units of pixels, the signal/background ratio becomes high, and if the antibody concentration is further lowered, the percentage of signals indicating 0 increases, and thus the signal/background ratio may also decrease.

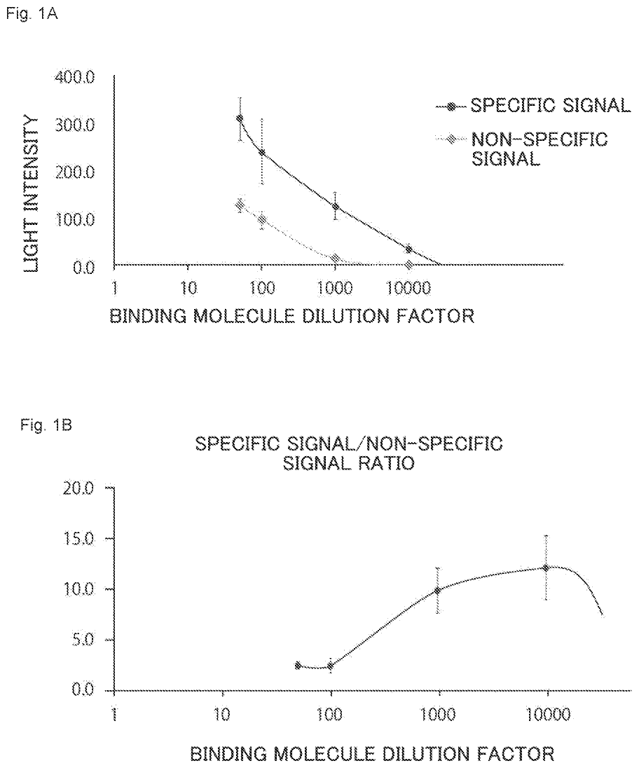

More specifically, graphs will be described as examples. For example, FIG. 1A is a graph showing the relationship between the binding molecule concentration and the light intensity, instead of drawings. FIG. 1B is a graph showing the relationship between the binding molecule concentration and a specific signal/non-specific signal ratio, instead of drawings. As shown in FIG. 1A, as the binding molecule concentration is lowered, the specific signal is reduced, but the non-specific signal is also reduced, and the non-specific signal approaches zero in units of pixels. Therefore, as shown in FIG. 1B, the specific signal/non-specific signal ratio increases. However, as shown in FIG. 1A, when the binding molecule concentration is further lowered, the percentage of specific signals indicating 0 increases in units of pixels, and accordingly, as shown in FIG. 1B, the specific signal/non-specific signal ratio decreases, and upward convex inflection points may be exhibited.

In this manner, the signal/background ratio changes depending on the concentration of the reagent and the like so that a large variation in detection accuracy may occur depending on the selection of the concentration of the reagent or the like. Therefore, if appropriate staining conditions for target molecules and a reagent containing binding molecules can be calculated, complicated operations performed by a user can be simplified and the detection accuracy can be improved.

2. Information Processing Device 1

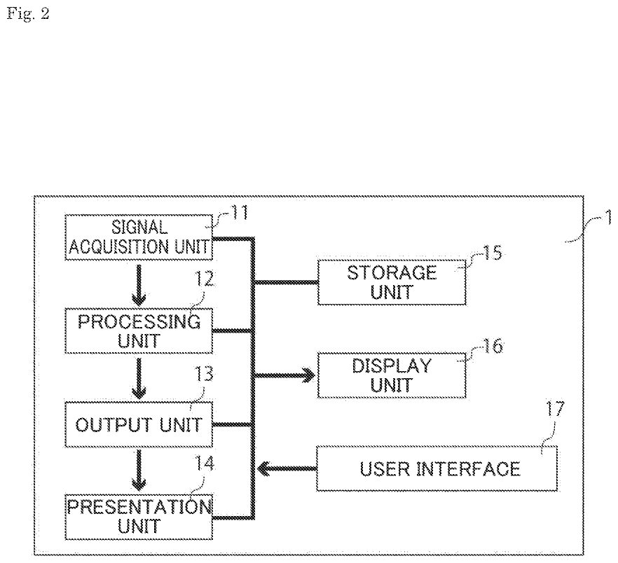

FIG. 2 is a block diagram showing an example of an information processing device 1 according to the present technology. The information processing device 1 according to the present technology includes at least a signal acquisition unit 11, a processing unit 12, and an output unit 13. In addition, it can include, as necessary, a presentation unit 14, a storage unit 15, a display unit 16, a user interface 17 and the like. Hereinafter, respective units and the like will be described in detail.

(1) Target Molecules

Target molecules in the present technology are molecules that can be detected and/or analyzed when they bind to binding molecules to be described below, and can be appropriately selected by those skilled in the art. Examples of target molecules include molecules that can be detected and/or analyzed when they bind to binding molecules in analysis such as flow cytometry, microscope observation, western blotting, various arrays, and ELISA. That is, the present technology can be used to support setting of reaction conditions with binding molecules used in these analyses.

More specifically, the target molecules are, for example, molecules existing in a living body, and examples thereof include biomolecules, drug molecules, and harmful molecules. Examples of biomolecules include nucleic acids, proteins, sugars, lipids, and vitamins. Examples of nucleic acids include DNA and RNA. Examples of proteins include antigen proteins, enzyme proteins, structural proteins, and adhesion proteins.

(2) Binding Molecules

In the present technology, the binding molecules are molecules that enable detection and/or analysis of target molecules when they bind to the above target molecules, and can be appropriately selected by those skilled in the art. Examples of binding molecules include molecules that enable detection and/or analysis of target molecules when they bind to target molecules in the above various analyses.

More specifically, the binding molecules are, for example, molecules existing in a living body, and examples thereof include biomolecules and drug molecules. Examples of biomolecules include nucleic acids, proteins, sugars, lipids, vitamins, and labeling molecules. Examples of nucleic acids include DNA and RNA. Examples of proteins include antibody proteins, cell surface markers, enzyme proteins, structural proteins, and adhesion proteins.

In addition, labeling molecules that label the binding molecules may be bound to the binding molecules. When the binding molecules to which the labeling molecules are bound bind to target molecules, the target molecules can be detected and/or analyzed. Examples of labeling molecules include molecules that can be used as labeling molecules in the above various analyses.

More specifically, the labeling molecules include, for example, a dye. Examples of dyes include various fluorescent dyes having fluorescence wavelengths in the visible light region, and examples thereof include fluorescent dyes of AlexaFluor (registered trademark) series, fluorescent dyes of DyLight (registered trademark) series, and fluorescent dyes of BD Horizon Brilliant (registered trademark) series, but the present technology is not limited thereto.

In addition, in the present technology, the labeling molecules may be expressed as a part of target molecules or binding molecules, and may be, for example, fluorescent proteins contained in fluorescent fusion proteins. Examples of fluorescent proteins include GFP, BFP, CFP, EGFP, EYFP, and PA-GFP.

A method of analyzing target molecules using an antibody to which a fluorescent dye is bound as a labeling molecule as binding molecules may be called fluorescent immunostaining. Fluorescent immunostaining includes, for example, immunocytochemistry (ICC) and immunohistochemistry (IHC). ICC is a method of staining cells isolated from tissues or cultured cells. IHC is a method of staining target molecules in thin sections of tissues.

In addition, the fluorescent immunostaining includes a direct fluorescent immunostaining method and an indirect fluorescent immunostaining method. The direct fluorescent immunostaining method is a method in which an antibody bound with a fluorescent dye directly binds to the target molecule, and the target molecule is analyzed by detecting the fluorescent dye. In the indirect fluorescent immunostaining method, an antibody bound with a fluorescent dye (also referred to as a “secondary antibody”) binds to an antibody specifically bound to the target molecule (also referred to as a “primary antibody”), which in turn binds to the target molecule. That is, the indirect fluorescent immunostaining method is a method in which an antibody bound with a fluorescent dye (also referred to as a “secondary antibody”) binds to the target molecule via the primary antibody, and the target molecule is analyzed by detecting the fluorescent dye.

In the present technology, when a secondary antibody is used, the processing unit 12 to be described below can calculate immunostaining conditions for the secondary antibody and the primary antibody. The present technology can be applied to both the direct fluorescent immunostaining method and the indirect fluorescent immunostaining method, but the direct fluorescent immunostaining method is preferable because there are few reaction steps, there is little variation, and quantification is improved.

The present technology can be suitably used for setting reaction conditions for the antigen-antibody reaction performed in fluorescent immunostaining.

(3) Signal Acquisition Unit 11

The signal acquisition unit 11 acquires a signal derived from a sample including a biological sample. For example, the signal acquisition unit 11 acquires signals detected by a flow cytometer, a microscope, various photodetectors and the like.

The signal acquisition unit 11 can acquire not only signals detected by various detection devices but also signal data in a database stored in the storage unit 15 to be described below. For example, the signal acquisition unit 11 can acquire past detection data, data detected by other detection devices and accumulated in a database, and the like.

The signal acquisition unit 11 can acquire fluorescence signals after autofluorescence separation and/or inter-dye color separation.

(4) Processing Unit 12

The processing unit 12 calculates immunostaining conditions of the reagent for the sample based on the signal acquired by the signal acquisition unit 11. The immunostaining conditions calculated in the present technology are conditions causing the immunostaining reaction to proceed, for example, treatment conditions such as an antibody clone, the concentration of binding molecules (antibodies), pH, the temperature, the antigen-antibody reaction time, antigen activation conditions, and stirring conditions, and the composition of the reaction solution, selection of the type of the buffer solution, enzyme treatment conditions, the ionic strength, the ion types, the ion concentration, stirring conditions and the like.

Here, the signals used for calculating immunostaining conditions include a signal derived from a reagent containing target molecules in the biological sample and binding molecules that can bind to the target molecules and/or a signal derived from non-target molecules and the reagent.

Here, in the present technology, the signal may be a fluorescence signal itself, a specific signal/background, a specific signal/non-specific signal or the like. In addition, the fluorescence signal may be in units of pixels such as signal/pixel, and as will be described below, cell segmentation is performed using morphology information (tissue, cell membrane, nuclear distribution, stroma, cytoplasm, etc.) determined based on the signal, and cell units such as fluorescence signal average/cells and fluorescence signal sum/cells can be obtained.

For example, when a histopathological specimen is used, the section thickness of the histopathological specimen is several micrometers. In particular, a section used for immunostaining having a thickness of about 4 micrometers is used, but the thickness of cells may be several micrometers to several tens of micrometers. Therefore, depending on the size of the cells contained in the section, the amount of target molecules contained changes, which may influence the staining signal obtained by immunostaining.

Therefore, when nuclear staining segmentation is performed and only staining signals derived from cells with a value equal to or larger than a certain level of nuclear staining signal are validated, it is possible to minimize a variation in the staining signal.

As a specific method of calculating the immunostaining conditions, for example, a method of calculating immunostaining conditions of the reagent for the sample including the biological sample based on the signals obtained when the binding molecule concentration (reagent concentration) is changed and the threshold value may be exemplified. That is, the processing unit 12 can calculate immunostaining conditions of the reagent for the sample including the biological sample based on the signals derived from the sample stained with a plurality of reagent concentrations and the threshold value.

In this case, regarding the threshold value, although a method of setting the threshold value of the luminance signal is not limited, for example, an average value of background signals of unstained specimen data or a value obtained by adding twice the standard deviation or three times the standard deviation of the average value may be used. In addition, the maximum value of the unstained specimen data, half the maximum value or the like may be used. In addition, it is also possible to use the average value of background signals arbitrarily selected from the stained specimen data, a value obtained by adding twice the standard deviation or three times the standard deviation, the maximum value, half the maximum value, or the like. In addition, the threshold value can be set for the stained specimen data using a P-tile method, a discriminant analysis method, a minimum error method, a differential histogram method, a Laplacian histogram method, a mean adjacent threshold determination method, a least complex binarization method, a moving average method, a region segmentation method or the like.

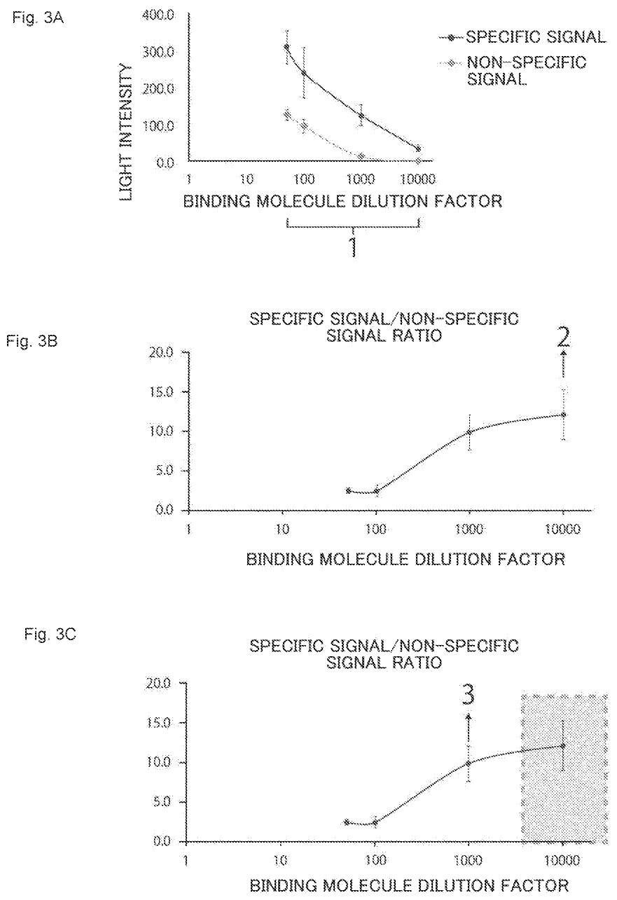

More specific examples will be described with reference to the drawings. FIG. 3A is a graph showing the relationship between the binding molecule concentration (reagent concentration) and the light intensity, instead of drawings. FIG. 3B and FIG. 3B′ are graphs showing the relationship between the binding molecule concentration (reagent concentration) and a specific signal/non-specific signal ratio, instead of drawings. The processing unit 12 first selects the binding molecule concentration (reagent concentration) that satisfies specific signal>non-specific signal (refer to reference number 1 in FIG. 3A). Next, it selects the binding molecule concentration (reagent concentration) at the inflection point of the specific signal/non-specific signal ratio (refer to reference number 2 in FIG. 3B). However, when the dilution factor of the binding molecules at the inflection point is 10,000, the value of the specific signal is low with reference to FIG. 3A. Therefore, the binding molecule concentration (reagent concentration) at which the value of the specific signal is sufficient and a specific signal/non-specific signal ratio is a maximum is selected (refer to reference number 3 in FIG. 3B′).

In addition, for example, when the signals obtained when the binding molecule concentration (reagent concentration) is changed are accumulated, and a calibration curve created from the accumulated signal data is referred to, signals obtained by reacting binding molecules with target molecules at different concentrations can be calculated from signals obtained by reacting binding molecules with target molecules at a single concentration.

That is, the processing unit 12 can calculate immunostaining conditions of the reagent for the sample including the biological sample based on the signal derived from the sample stained with at least one reagent concentration and the reagent information referred to in the database.

In this manner, with reference to a calibration curve created from the accumulated signal data, it is not necessary to measure the binding molecule concentration, and by simply reacting binding molecules and target molecules at a single concentration, signals obtained when binding molecules at different concentrations are used can be calculated. Here, in this case, a calibration curve of each antibody clone or fluorescence-labeled antibody (clone) may be referred to from the database.

As described above, in the present technology, immunostaining conditions can be calculated, but when the percentage of signals indicating zero increases, there is a problem that the region or area that should be inherently detected decreases and region information and morphology information that should be detected are not obtained.

Therefore, the processing unit 12 can calculate immunostaining conditions of the reagent for the sample including the biological sample based on the signals derived from the sample stained with a plurality of reagent concentrations and the threshold value extracted from region information.

The processing unit 12 can determine the region based on the signal and/or bright field image. The region can also include morphology information (tissue, cell membrane, nuclear distribution, stroma, cytoplasm, etc.) of the biological sample. A method of determining the region is not particularly limited, and a nuclear distribution can be determined by staining cell nuclei using binding molecules for staining cell nuclei, for example, 4′,6-diamidino-2-phenylindole (DAPI), Hoechst, or propidium iodide (PI), and the morphology such as the cell membrane or cytoplasm can be determined using binding molecules for detecting target molecules present in the cell membrane, cytoplasm or the like.

If the value of the specific signal is sufficient, satisfactory extraction of the region can be performed based on the specific signal. For example, when membrane staining is performed, it can be determined that it is sufficient if the membrane staining is continuous at a certain level or more, and when nuclear staining is performed, it can be determined that it is sufficient if the area of nuclear staining is a certain level or more.

In this manner, if the region in the sample is determined, visual information can be provided for a user. Specifically, for example, when a nuclear distribution is determined by staining cell nuclei, it is easier to analyze the cell unit, and when the region (morphology) such as the cell membrane or cytoplasm is determined by staining the cell membrane or cytoplasm, it is easier to distinguish whether it is the cell membrane. In addition, when the region in the sample is determined, quantification can be improved, and it is easier to distinguish between specific staining and non-specific staining.

As a method of extracting the threshold value from region information, labeling can be performed using different binding molecules, and the ratio of the labeled region to that can be used. Hereinafter, specific examples will be described.

<Nuclear Staining>

A method of calculating staining conditions with labeled antibodies used for detection when target molecules present in the nucleus region such as Ki-67, which is a breast cancer proliferation-related gene, progesterone receptors, estrogen receptors and the like are detected may be exemplified.

First, in order to extract the nucleus region (morphology), morphology information (region) of nuclei is determined by staining cell nuclei using binding molecules for staining cell nuclei such as 4′,6-diamidino-2-phenylindole (DAPI), Hoechst, or propidium iodide (PI).

A percentage of signals obtained from binding molecules such as labeled antibodies used for detecting target molecules present in the nucleus region such as Ki-67, progesterone receptors, and estrogen receptors within the determined nucleus region is calculated. Staining conditions in which a percentage thereof is a certain level or more are validated.

<Cell Membrane>

A method of calculating staining conditions with labeled antibodies used for detection when target molecules present in the cell membrane of T cells such as CD8 and CD4 are detected may be exemplified.

First, molecules (for example, CD3, etc.) different from the target molecules present in the cell membrane of T cells are stained with labeled antibodies different from the labeled antibodies used for detecting the target molecules, and the region (morphology) of the cell membrane is determined.

A percentage of signals obtained from binding molecules such as labeled antibodies used for detecting target molecules present in the cell membrane of T cells such as CD8 and CD4 within the determined cell membrane region is calculated. Staining conditions in which a percentage thereof is a certain level or more are validated.

<Cytoplasm>

A method of calculating staining conditions with labeled antibodies used for detection when target molecules present in the cytoplasm such as cytokeratin are detected may be exemplified.

First, molecules present in the cytoplasm such as tubulin, GAPDH (Glyceraldehyde-3-phosphate dehydrogenase), and actin are stained using labeled antibodies different from labeled antibodies used for detecting target molecules or using binding molecules such as phalloidin, and the region (morphology) of the cytoplasm is determined.

A percentage of signals obtained from binding molecules such as labeled antibodies used for detecting target molecules present in the cytoplasm such as cytokeratin within the determined cytoplasm region is calculated. Staining conditions in which a percentage thereof is a certain level or more are validated.

The processing unit 12 can also compare the plurality of determined regions. In addition, the processing unit 12 can compare the plurality of regions including at least one of the cell membrane, the cell nucleus, a specific binding region and a non-specific binding region, and thus can analyze localization of the regions. For example, as described above, cell nuclei are stained using binding molecules (4′,6-diamidino-2-phenylindole (DAPI), Hoechst, propidium iodide (PI), etc.) for staining cell nuclei, the plurality of stained regions are compared, and thus the position of the nuclei can be determined.

Here, an observation method for determining the region is not particularly limited, and may be any of bright field observation, dark field observation and oblique illumination observation. In addition, the image used for determining the region may be a raw image, but an image that has undergone image processing can also be used. For image processing, a signal may be excluded using a luminance signal threshold value, and an image from which a signal is extracted, a multivalued image, a binarized image or the like may be used. In addition, a specific staining evaluation algorithm may be applied from the staining morphology of the image after autofluorescence separation or fluorescent dye color separation. In addition, an image converted to the number of binding molecules (antibodies) in place of the light intensity may be used. When an image converted to the number of binding molecules (antibodies) is used, it is also possible to compare binding molecules using different dyes or the like.

A method of setting the threshold value of the luminance signal is not limited, and for example, an average value of background signals of unstained specimen data or a value obtained by adding twice the standard deviation or three times the standard deviation of the average value may be used. In addition, the maximum value of the unstained specimen data, half the maximum value or the like may be used. In addition, it is also possible to use the average value of background signals arbitrarily selected from the stained specimen data, a value obtained by adding twice the standard deviation or three times the standard deviation, the maximum value, half the maximum value, or the like. In addition, the threshold value can be set for the stained specimen data using a P-tile method, a discriminant analysis method, a minimum error method, a differential histogram method, a Laplacian histogram method, a mean adjacent threshold determination method, a least complex binarization method, a moving average method, a region segmentation method or the like.

The region extraction method is not particularly limited, and for example, a region extraction according to feature amount extraction, region extraction according to defect complementation or the like can be appropriately selected. In addition, the region and morphology (tissue, cell membrane, nuclear distribution, stroma, cytoplasm, etc.) extracted according to template matching, pattern recognition, or image segmentation can be used. Specifically, for example, an image gradient is calculated and a threshold value is calculated so that a binary mask including the segmented regions is created. As necessary, the binary mask region is adjusted using structuring elements or the like. As necessary, a process of filling blank regions present in the mask region is performed. If there are other objects or debris in contact with segmented objects, the connectivity of pixels can be evaluated, and increased, decreased, or exclusion can be performed by weighting. As necessary, structuring elements can be used to smooth the mask region. When the mask region is applied to the original image, the region can be extracted.

For example, the inside of the mask region can be defined as a signal derived from a reagent containing binding molecules that can bind to target molecules or a specific staining region, and the other region can be defined as a non-target molecule, a signal derived from the reagent, or a non-specific staining region. In some cases, by inverting the inside and outside of the mask region, the inside of the mask region can be used as a non-target molecule, a signal derived from the reagent or a non-specific staining region. In addition, for the signal derived from the reagent containing binding molecules that can bind to target molecules or the specific staining region, and the non-target molecules, the signal derived from the reagent, or the non-specific staining region, separate masks can be created for respective regions, and respective regions can be independently extracted.

The present technology can be used in analysis such as flow cytometry, microscope observation, western blotting, various arrays, and ELISA, but for example, in microscope observation, when a histopathological specimen is used, there are cases in which cells are adjacent to each other, such as tumor cells in tumor tissue, T cells present in cortex in lymphoid tissue, or B cells in follicles. Since the cell membranes in the border region in which cells are adjacent to each other overlap, they exhibit a larger signal value than the cell membrane in which cells exist alone. In this manner, a mixture of overlapping cell membranes and single cell membrane causes variation in data.

Therefore, when the plurality of determined regions are single cells, the processing unit 12 can exclude signals derived from single cells that overlap and/or are adjacent to each other within a predetermined distance and then calculate the area staining conditions.

More specifically, cells exhibiting cell membrane staining signals and whose center-to-center distance between cells is equal to or larger than the size of the cells are validated, the inside of the staining signal of cell membrane staining is repaired, the distribution of the repaired area is calculated, the smallest area larger than the area of nuclear staining such as DAPI is defined as single cells, only the staining signal derived from single cells is validated, aggregates of a plurality of cells are excluded, and the immunostaining conditions can then be calculated. In this manner, when signals derived from single cells that overlap and/or are adjacent to each other within a predetermined distance are excluded, for example, it is possible to minimize a variation in the staining signal caused by an increase in signals due to overlapping cell membranes.

In addition, when a histopathological specimen is used, as described above, depending on the size of the cells contained in the section, the amount of target molecules contained changes, which may influence the staining signal obtained by immunostaining.

Therefore, the processing unit 12 validates a certain level or more of the total luminance or total area of cell nuclei staining (DAPI, etc.) or a certain percentage or more of the upper part of the distribution of the total luminance and the total area of nuclear staining per cell in the captured image, and then can calculate the immunostaining conditions. As a result, it is possible to minimize a variation in the staining signal.

(5) Output Unit 13

The information processing device 1 according to the present technology includes the output unit 13 that outputs information processed by the processing unit 12. Specifically, the output unit 13 can output various types of information related to the calculated immunostaining conditions and presenting of immunostaining conditions such as the set threshold value and also various types of information related to detection performed for presenting the immunostaining conditions.

More specifically, the output unit 13 can output, as the immunostaining conditions, at least one of the antibody clone, the antibody concentration, the antigen-antibody reaction time, the reaction temperature, the antigen activation condition, the composition of the reaction solution and stirring conditions.

(6) Presentation Unit 14

The presentation unit 14 presents support information for staining conditions to the user based on the immunostaining conditions output from the output unit 13. In the past, since staining conditions were directly set by the user, the staining conditions were not appropriate in some cases, and even for the experienced user, it was a very time-consuming task. However, according to the present technology, based on the immunostaining conditions calculated by the processing unit 12 and output from the output unit 13, for example, since optimal immunostaining conditions are presented according to the type of target molecules to be detected and analyzed, the state of the sample, and the like, it is possible to perform detection with high accuracy regardless of the user's experience.

In the present technology, the presentation unit 14 is not essential, and a staining device or the like can automatically stain the sample without intervention of the user. For example, the immunostaining conditions output from the output unit 13 are directly output to various staining devices, and based on the immunostaining conditions acquired by various staining devices, the various staining devices can automatically stain the sample.

(7) Storage Unit 15

The information processing device 1 according to the present technology can include the storage unit 15 that stores various pieces of information. The storage unit 15 can accumulate and store any data such as information processed by the processing unit 12, various types of information related to presentation of immunostaining conditions such as various signals and various threshold values, and also various types of information related to detection for presenting immunostaining conditions.

In the information processing device 1 according to the present technology, the storage unit 15 is not essential, and each piece of information can be output from the output unit 13 to the outside of the device, and can be stored in an external storage device 21 to be described below. The storage device 21 can also be provided in a cloud environment and can be connected to the information processing device 1 according to the present technology via a network. In this case, various pieces of information stored in the storage device 21 on the cloud can be shared by a plurality of users.

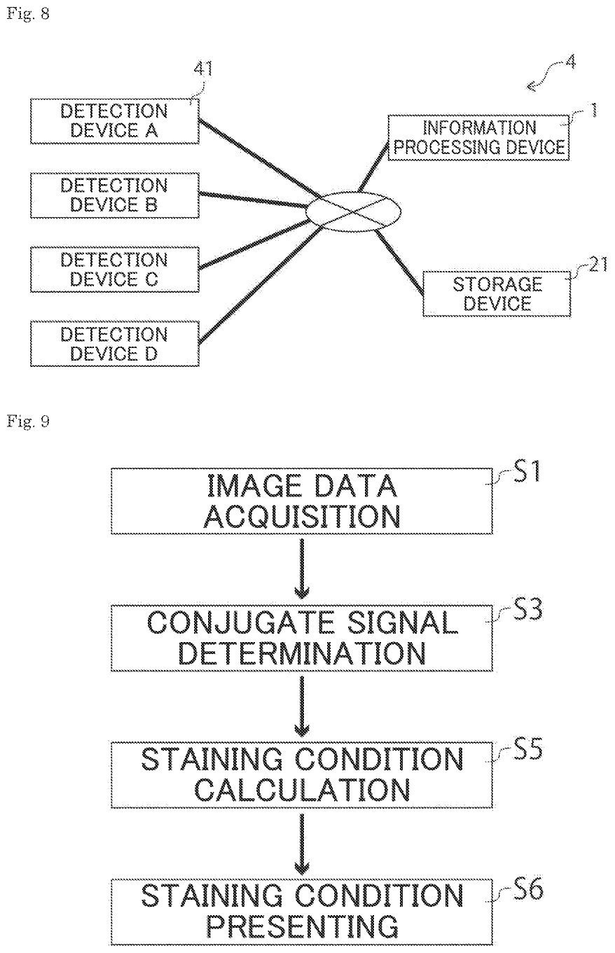

In the storage unit 15 and the external storage device 21, a database can be constructed based on information output from the output unit 13, as shown in FIG. 8 described below, detection data detected by external detection devices 41A to 41D, and the like, detection data collected in other samples and the like. In this case, the processing unit 12 can perform various types of processing with reference to the database.

For example, with reference to data implemented in the past, detection data detected by the external detection devices 41A to 41D and the like, detection data collected in other samples and the like, without the user directly performing actual measurement, it is possible to calculate reaction conditions between target molecules and binding molecules, and set the threshold value.

In addition, for example, a calibration curve is created from the signal data accumulated in the database, and with reference to this, immunostaining conditions can be optimized by fitting measurement data with a single concentration to the calibration curve without performing measurement with a plurality of concentrations (reagent concentrations) of binding molecules.

(8) Display Unit 16

The information processing device 1 according to the present technology can include the display unit 16 that displays various pieces of information output from the output unit 13. As the display unit 16, for example, a general display device such as a display or a printer can be used.

(9) User Interface 17

The information processing device 1 according to the present technology can further include the user interface 17 for user operation. The user can access respective units and control respective units through the user interface 17.

In the present technology, the user interface 17 is not essential, and an external operation device may be connected. As the user interface 17, for example, a mouse, a keyboard or the like can be used.

<3. Information Processing System 2>

FIG. 4 is a conceptual diagram showing an example of the information processing system 2 according to the present technology. The information processing system 2 according to the present technology includes the information processing device 1 of the present technology and the storage device 21 that stores information processed by the information processing device 1. In addition, the information processing system 2 according to the present technology can include, as necessary, a display device 22, a user interface 23 and the like. Since details of the information processing device 1 are the same as details of the information processing device 1 of the present technology described above, descriptions thereof are omitted here. In addition, since details of the storage device 21, the display device 22, and the user interface 23 are the same as details of the storage unit 15, the display unit 16, and the user interface 17 of the information processing device 1 of the present technology described above, descriptions thereof are omitted here.

<4. Target Molecule Detection Device 3 and Target Molecule Detection System 4>

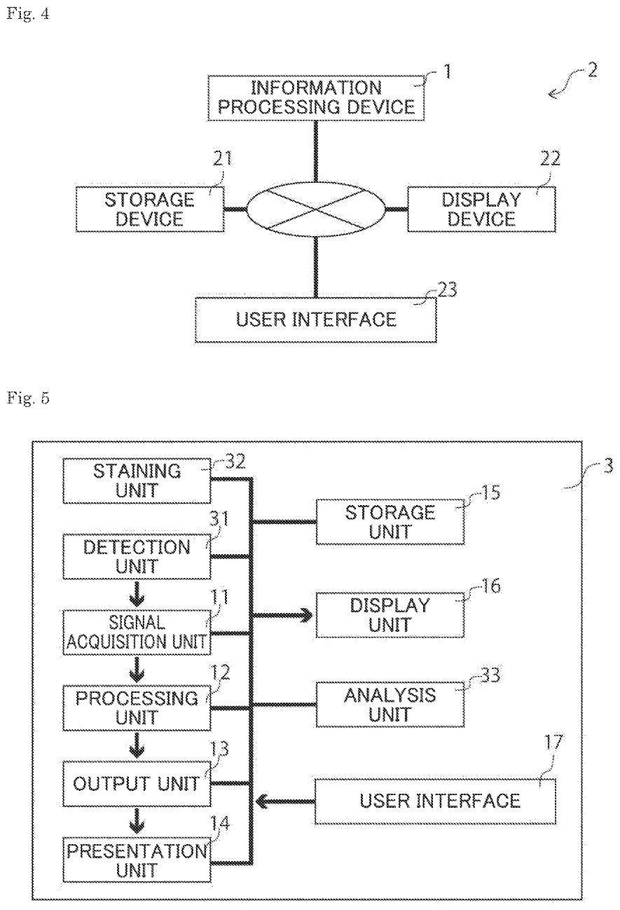

FIG. 5 is a block diagram showing an example of the target molecule detection device 3 according to the present technology. The target molecule detection device 3 according to the present technology includes a detection unit 31 that detects a signal derived from a sample including a biological sample, the signal acquisition unit 11, the processing unit 12, and the output unit 13. In addition, the target molecule detection device 3 according to the present technology can include, as necessary, the presentation unit 14, the storage unit 15, the display unit 16, the user interface 17, a staining unit 32, an analysis unit 33 and the like. Since details of the signal acquisition unit 11, the processing unit 12, the output unit 13, the presentation unit 14, the storage unit 15, the display unit 16, and the user interface 17 are the same as details of the signal acquisition unit 11, the processing unit 12, the output unit 13, the presentation unit 14, the storage unit 15, the display unit 16, and the user interface 17 of the information processing device 1 described above, descriptions thereof are omitted here.

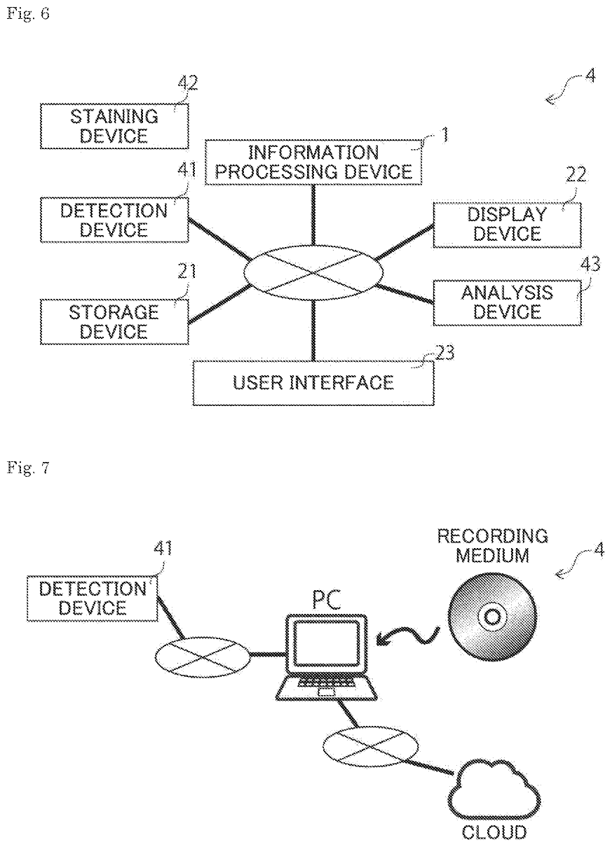

FIG. 6 is a conceptual diagram showing an example of the target molecule detection system 4 according to the present technology. The target molecule detection system 4 according to the present technology includes the detection device 41 that detects a signal derived from a sample including a biological sample and the information processing device 1 according to the present technology described above. In addition, the target molecule detection system 4 according to the present technology can include, as necessary, the storage unit 15, the storage device 21, the display device 22, the user interface 23, the staining unit 32, a staining device 42, the analysis unit 33, an analysis device 43 and the like. Since details of the information processing device 1 are the same as details of the information processing device 1 described above, descriptions thereof are omitted here. In addition, since details of the storage unit 15, the storage device 21, the display device 22, and the user interface 23 are the same as details of the storage unit 15, the display unit 16, and the user interface 17 of the information processing device 1 of the present technology described above, descriptions thereof are omitted here.

FIG. 7 is a conceptual diagram showing an example of the target molecule detection system 4 according to the present technology different from FIG. 6. The target molecule detection system 4 according to the present technology includes the detection device 41 that detects a signal derived from a sample including a biological sample and a computer program to be described below.

(1) Detection Unit 31 and Detection Device 41

The detection unit 31 and the detection device 41 detect a signal derived from a sample including a biological sample. In addition, the detection unit 31 and the detection device 41 can detect a signal derived from the stained sample based on the immunostaining condition information output from the output unit 13. As the detection unit 31 and the detection device 41 that can be used in the present technology, a general detection unit 31 and detection device 41 can be freely used as long as they can detect a signal emitted from a sample including a biological sample. For example, the detection unit 31 and the detection device 41 that can be used in analysis such as flow cytometry, microscope observation, western blotting, various arrays, and ELISA may be exemplified.

In the target molecule detection system 4 according to the present technology, the information processing device 1 and/or the storage device 21 can be provided in a cloud environment and can be connected to the detection device 41 via a network. In this case, various pieces of information stored in the storage device 21 on the cloud can be shared by a plurality of users. Specifically, for example, as shown in FIG. 8, the plurality of detection devices 41A to 41D are connected to the information processing device 1 and/or the storage device 21 via a network, and using the signals detected by the plurality of detection devices 41A to 41D, the information processing device 1 performs processing, and the immunostaining conditions output from the information processing device 1 can be shared by the plurality of detection devices 41A to 41D.

(2) Staining Unit 32 and Staining Device 42

The staining unit 32 and the staining device 42 stain a sample including a biological sample using a reagent containing binding molecules that can bind to target molecules in the biological sample. The staining unit 32 and the staining device 42 can stain the sample under optimal conditions based on the immunostaining conditions output from the output unit 13. As a result, it is possible to improve the accuracy of target molecule detection.

Here, the staining unit 32 and the staining device 42 are not essential in the target molecule detection device 3 and the target molecule detection system 4 according to the present technology, and it is possible to stain the sample using an external staining device or the like based on the immunostaining conditions output from the output unit 13.

(3) Analysis Unit 33 and Analysis Device 43

The analysis unit 33 and the analysis device 43 analyze the sample based on the signals detected by the detection unit 31 and the detection device 41. More specifically, it is possible to analyze the type of cells contained in the sample, the amount and properties thereof, and the like based on the signals detected by the detection unit 31 and the detection device 41.

Here, the analysis unit 33 and the analysis device 43 are not essential in the target molecule detection device 3 and the target molecule detection system 4 according to the present technology, and it is also possible to analyze properties of target molecules in the sample using an external analysis device or the like based on the signals detected by the detection unit 31 and the detection device 41. For example, the analysis unit 33 and the analysis device 43 may be implemented in a personal computer or a CPU or may be stored as a program in hardware resources including a recoding medium (for example, a nonvolatile memory (a USB memory), an HDD, a CD, etc.) and the like, and can be operated by a personal computer or a CPU. In addition, the analysis unit 33 and the analysis device 43 may be connected to respective units of the target molecule detection device 3 and the target molecule detection system 4 via a network.

<5. Computer Program>

A computer program according to the present technology is a program causing a computer to implement a signal acquisition function of acquiring a signal derived from a sample including a biological sample, a processing function of calculating immunostaining conditions of the reagent for the sample including the biological sample based on the signal, and an output function of outputting the immunostaining conditions.

The computer program according to the present technology is recorded in an appropriate recoding medium. In addition, the computer program according to the present technology can be stored in a cloud environment or the like, and the user can download it to a personal computer or the like via a network and use it. Here, since the signal acquisition function, the processing function, and the output function in the computer program according to the present technology are the same as functions performed by the signal acquisition unit 11, the processing unit 12, and the output unit 13 of the information processing device 1 described above, descriptions thereof are omitted here.

<6. Information Processing Method>

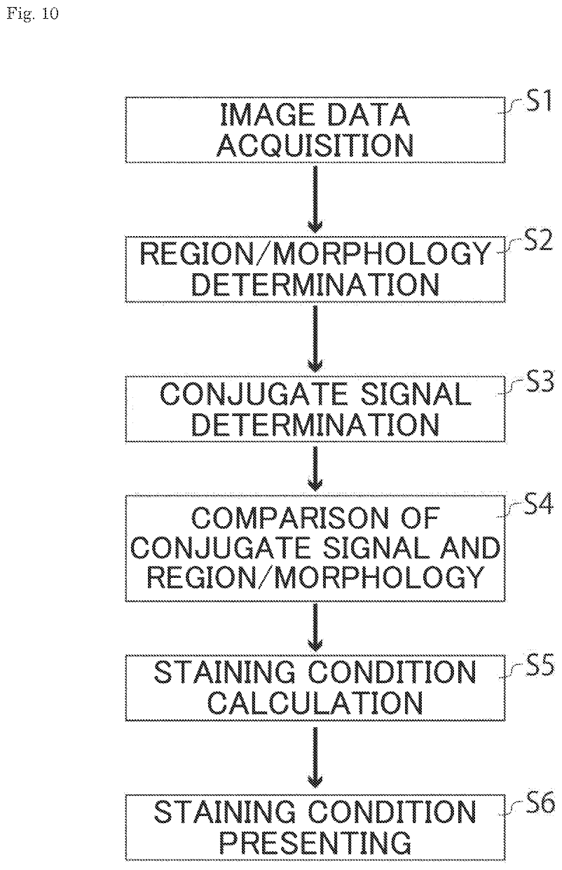

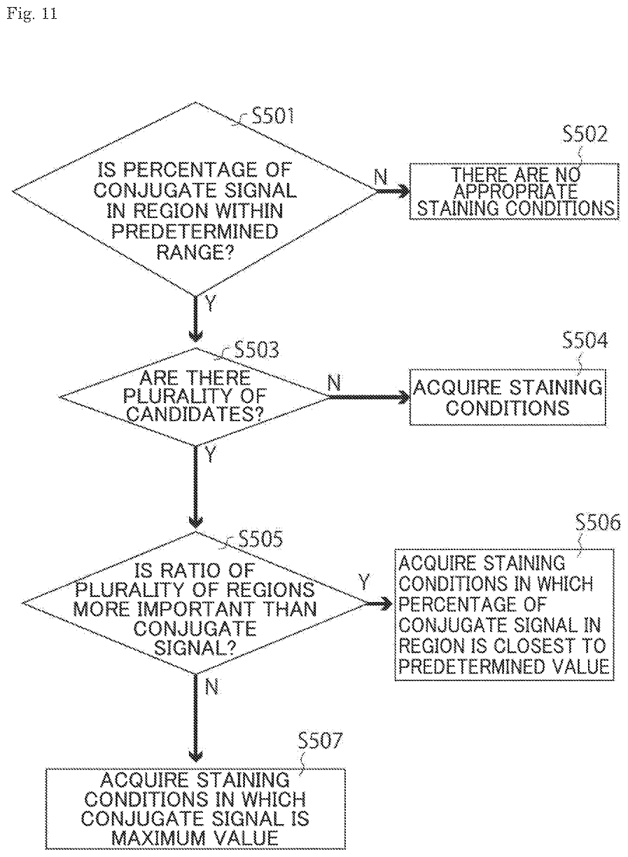

FIG. 9 is a flowchart showing an example of an information processing method according to the present technology. FIG. 10 is a flowchart showing an example of the information processing method according to the present technology different from FIG. 9. In the information processing method according to the present technology, at least image data acquisition S1 (signal acquisition step), conjugate signal determination S3 and immunostaining condition calculation S5 (processing step), and immunostaining condition presenting S6 (output step) are performed. In addition, as necessary, region/morphology determination S2, and comparison of conjugate signal and region/morphology S4 can be performed. Hereinafter, respective steps will be described in detail in time series. Here, in embodiments shown below, a method of presenting immunostaining conditions for an anti-progesterone receptor (hereinafter referred to as “PgR”) antibody will be exemplified. An anti-PgR antibody is an antibody that specifically stains nuclei under appropriate immunostaining conditions.

(1) Image Data Acquisition S1

First, image data obtained by the detection unit 31 and/or the detection device 41 described above is acquired. More specifically, each piece of image data immunostained with one or more antibody concentrations is acquired from the detection unit 31 and/or the detection device 41 described above. In this case, as necessary, it is also possible to acquire image data at concentrations with antibodies not included and image data immunostained with various isotype antibodies.

(2) Region/Morphology Determination S2

As shown in the modified example in FIG. 10, it is possible to determine a predetermined region and a predetermined morphology from the acquired image data. Specifically, cell nuclei are stained using binding molecules for staining cell nuclei, for example, 4′,6-diamidino-2-phenylindole (DAPI), Hoechst, or propidium iodide (PI), to determine the region/morphology of nuclei, and the region/morphology of the cell membrane, the cytoplasm and the like are determined using binding molecules for detecting target molecules present in the cell membrane, the cytoplasm and the like.

A method of determining the region/morphology is as described in the processing unit 12 described above. As a specific example, for example, when the threshold value of the DAPI staining signal is set and the positive region of the DAPI staining signal is extracted, it is possible to determine the region/morphology of nuclei. In addition, after the positive region of the DAPI staining signal is extracted, as necessary, image segmentation can be performed. In addition, when the region/morphology is determined, the images accumulated in the database can be referred to.

(3) Conjugate Signal Determination S3

Next, from the acquired image data, signals from conjugates of target molecules and binding molecules and/or signals from conjugates of non-target molecules and binding molecules are determined. A method of determining the signal is as described in the processing unit 12 described above.

As a specific example, for example, the threshold value of the PgR staining signal is set, the positive region of the PgR staining signal is extracted, and thus conjugate signals are determined. When the threshold value of the PgR staining signal is set, the threshold value of immunostaining for PgR can be set according to the concentration without anti-PgR antibodies or the staining signal of immunostaining of isotype antibodies of anti-PgR antibodies. In addition, when an image including positive cells and negative cells in one specimen is used, the threshold value of immunostaining for PgR can be set according to the PgR immunostaining signal for negative cells.

When a conjugate signal is determined, the signal may be the signal itself, a signal/background ratio, a specific signal/non-specific signal ratio or the like. As a specific example, for example, based on the set threshold value of the PgR staining signal, it is possible to specify a positive region (signal) that is equal to or more than the threshold value and a negative region (background) that is equal to or less than the threshold value, and it is possible to calculate the signal/background of the PgR staining signal.

(4) Comparison of Conjugate Signal and Region/Morphology S4

As shown in the modified example in FIG. 10, when the region/morphology determination S2 is performed, comparison with the determined conjugate signal is performed. Since the conjugate signal acquired from the outside of the determined region/morphology cannot be used for calculation of staining conditions, the conjugate signal within the determined region/morphology is extracted.

As a specific example, for example, a percentage of the PgR-positive region in the DAPI-positive region (PgR-positive region/DAPI-positive region), the value of the PgR staining signal in the region segmented with DAPI, a percentage of the PgR-positive region in the segmented DAPI-positive region (PgR-positive region/segmented DAPI-positive region) and the like are calculated.

(5) Immunostaining Condition Calculation S5

Based on the determined conjugate signal, immunostaining conditions of the reagent containing target molecules and binding molecules are calculated. Specifically, staining conditions in which the determined conjugate signal (signal/background) is the maximum value are acquired, as shown in FIG. 3 described above, when there is an inflection point in the relationship between the antibody concentration and the staining signal, staining conditions at the inflection point are acquired.

In addition, when the region/morphology determination S2 is performed, immunostaining conditions in which the conjugate signal (signal/background) in the determined region/morphology is closest to the maximum value or a predetermined value are acquired. A specific example of the staining condition calculation S5 when the region/morphology determination S2 is performed will be described with reference to a flowchart in FIG. 11.

FIG. 11 is a flowchart showing a specific example of immunostaining condition calculation S5 when region/morphology determination S2 in the information processing method using the present technology is performed. First, it is determined whether the percentage of the conjugate signal in the region is within a predetermined range (S501). As a specific example, for example, it is determined whether the percentage of the PgR-positive region in the DAPI-positive region is within a predetermined range (S501).

When the percentage of the conjugate signal in the region is not within a predetermined range, it is determined that there are no appropriate staining conditions (S502). When the percentage of the conjugate signal in the region is within a predetermined range, it is determined whether there are a plurality of candidates (S503). When the candidates are narrowed down to one, its staining conditions are acquired (S504). When there are a plurality of candidates, it is determined whether the ratio of the plurality of regions is more important than the conjugate signal (S505).

When the ratio of the plurality of regions is more important than the conjugate signal, staining conditions in which the percentage of the conjugate signal in the region is closest to the predetermined value are acquired (S506). In this case, when the staining conditions cannot be significantly narrowed down to one, among staining conditions in which the percentage of the conjugate signal in the region is closest to the predetermined value, staining conditions in which the conjugate signal is the maximum value can be acquired.

On the other hand, when the ratio of the plurality of regions is less important than the conjugate signal, staining conditions in which the conjugate signal is the maximum value are acquired (S507). In this case, when staining conditions cannot be significantly narrowed down to one, among staining conditions in which the conjugate signal is the maximum value, staining conditions in which the percentage of the conjugate signal in the region is closest to the predetermined value can be acquired.

Here, for the conjugate signal in FIG. 11, the signal/background can also be used. In addition, appropriately, various determinations can be performed with reference to the information accumulated in the database.

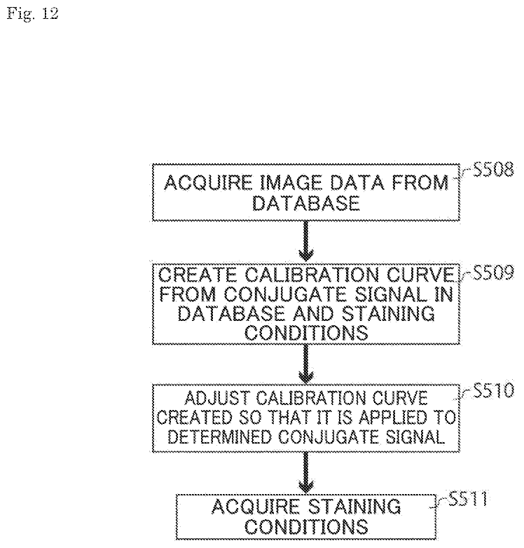

In the immunostaining condition calculation S5, as shown in FIG. 12, based on the information accumulated in the database, it is also possible to calculate staining conditions using a calibration curve or the like. FIG. 12 is a flowchart showing a specific example of a case in which, in the information processing method using the present technology, staining condition calculation S5 is performed using the information accumulated in the database.

First, image data is acquired from the database (S508), and a calibration curve is created from the information about the conjugate signals accumulated in the database and staining conditions (S509). Adjustment is performed so that the created calibration curve is applied to the determined conjugate signal (S510). Staining conditions are acquired from the adjusted calibration curve (S511).

(6) Immunostaining Condition Presenting S6

Based on the immunostaining conditions calculated according to the immunostaining condition calculation S5, support information for staining conditions is presented to the user. In the past, since staining conditions were directly set by the user, the staining conditions were not appropriate, and even for the experienced user, it was a very time-consuming task. However, according to the present technology, based on the immunostaining conditions calculated according to the immunostaining condition calculation S5, for example, since optimal immunostaining conditions are presented according to the type of target molecules to be detected and analyzed, the state of the sample, and the like, it is possible to perform detection with high accuracy regardless of the user's experience.

Hereinafter, a flow of embodiments according to the present technology will be described with reference to specific examples.

### First Embodiment

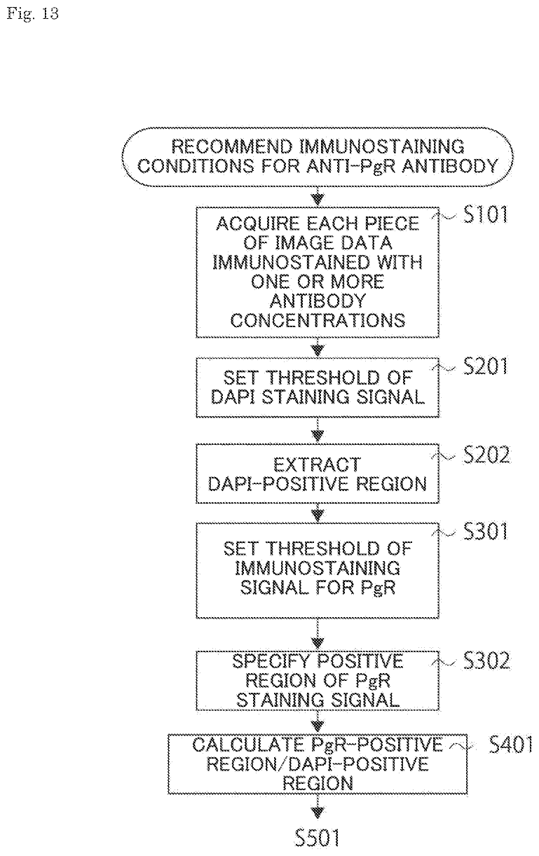

FIG. 13 to FIG. 16 are flowcharts showing a first embodiment of a method of presenting immunostaining conditions for anti-PgR antibodies using the information processing device according to the present technology.

Antibody conditions: conditions with one or more antibody concentrations

Specimen: when the specimen contains only positive cells

Staining signal: value of fluorescence signal

Database reference: none

First, each piece of image data immunostained with one or more antibody concentrations is acquired (S101).

Next, the threshold value of the DAPI staining signal is set (S201), and the positive region of the DAPI staining signal is extracted (S202).

Next, the threshold value of the PgR staining signal is set (S301), and the positive region of the PgR staining signal is specified (S302).

A percentage of the PgR-positive region in the DAPI-positive region (PgR-positive region/DAPI-positive region) is calculated (S401).

When the percentage of the PgR-positive region in the DAPI-positive region is not within a predetermined range (S501), it is determined that that there are no appropriate immunostaining conditions (S502), and the process returns, and when the percentage of the PgR-positive region in the DAPI-positive region is within a predetermined range (S501), and when there are no plural candidates (S503), immunostaining conditions are acquired (S504), and the acquired immunostaining conditions are presented (S600).

When the percentage of the PgR-positive region in the DAPI-positive region is within a predetermined range and there are a plurality of candidates (S503), and when the ratio of the plurality of regions is more important than the staining signal (S505), immunostaining conditions in which the percentage of the PgR-positive region in the DAPI-positive region is closest to an arbitrary value are acquired (S506). When the acquired immunostaining conditions are significantly narrowed down to one (S507), immunostaining conditions are acquired (S508), and the acquired immunostaining conditions are presented (S601). When the acquired immunostaining conditions are not significantly narrowed down to one (S507), staining conditions in which the PgR immunostaining signal is the maximum value are acquired (S509), and the acquired immunostaining conditions are presented (S602).

When the percentage of the PgR-positive region in the DAPI-positive region is within a predetermined range and there are a plurality of candidates (S503), and when the ratio of the plurality of regions is less important than the staining signal (S505), staining conditions in which the PgR immunostaining signal is the maximum value are acquired (S510). When the acquired immunostaining conditions are significantly narrowed down to one (S511), immunostaining conditions are acquired (S512), and the acquired immunostaining conditions are presented (S603). When the acquired immunostaining conditions are not significantly narrowed down to one (S511), immunostaining conditions in which the percentage of the PgR-positive region in the DAPI-positive region is closest to an arbitrary value are acquired (S513), and the acquired immunostaining conditions are presented (S604).

### Second Embodiment

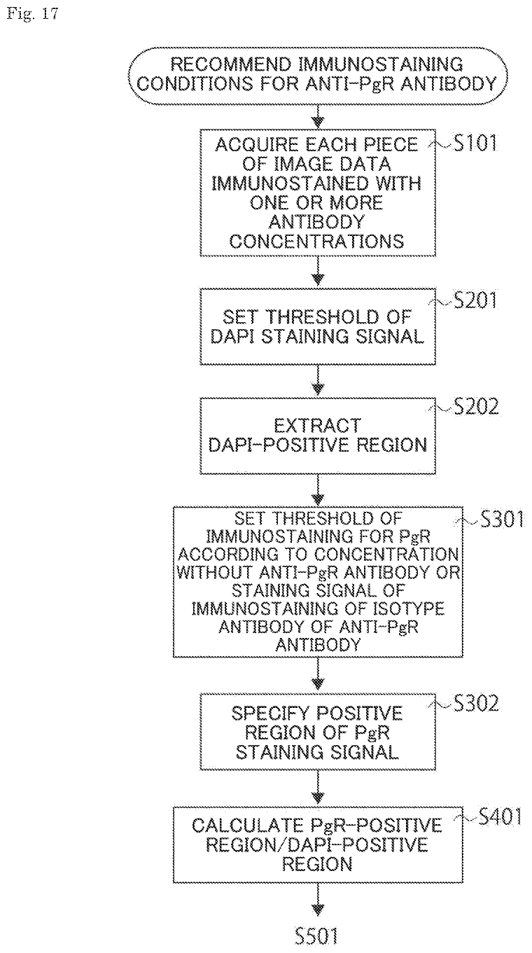

FIG. 17 is a flowchart showing a second embodiment of the method of presenting immunostaining conditions for anti-PgR antibodies using the information processing device according to the present technology.

Antibody conditions: conditions with one or more antibody concentrations, concentration with antibodies not included, and isotype antibodies included

Specimen: when the specimen contains only positive cells

Staining signal: value of fluorescence signal

Database reference: none

First, each piece of image data immunostained with one or more antibody concentrations is acquired (S101).

Next, the threshold value of the DAPI staining signal is set (S201), and the positive region of the DAPI staining signal is extracted (S202).

Next, the threshold value of immunostaining for PgR is set according to the concentration without anti-PgR antibodies or the staining signal of immunostaining of isotype antibodies of anti-PgR antibodies (S301), and the positive region of the PgR staining signal is specified (S302).

The steps after this are the same as S401 to S604 of the first embodiment shown in FIG. 13 to FIG. 16.

### Third Embodiment

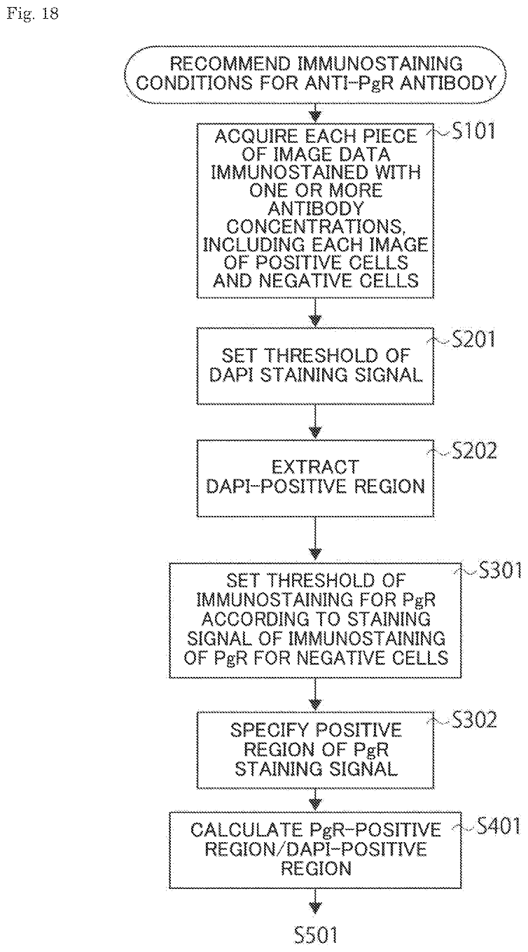

FIG. 18 is a flowchart showing a third embodiment of the method of presenting immunostaining conditions for anti-PgR antibodies using the information processing device according to the present technology.

Antibody conditions: conditions with one or more antibody concentrations

Specimen: when an image in which the specimen contains only positive cells and an image in which the specimen contains only negative cells are used.

Staining signal: value of fluorescence signal

Database reference: none

First, each piece of image data immunostained with one or more antibody concentrations (including image data of positive cells and negative cells) is acquired (S101).

Next, the threshold value of the DAPI staining signal is set (S201), and the positive region of the DAPI staining signal is extracted (S202).

Next, the threshold value of immunostaining for PgR is set according to the PgR immunostaining signal for negative cells (S301), and the positive region of the PgR staining signal is specified (S302).

The steps after this are the same as S401 to S604 of the first embodiment shown in FIG. 13 to FIG. 16.

### Fourth Embodiment

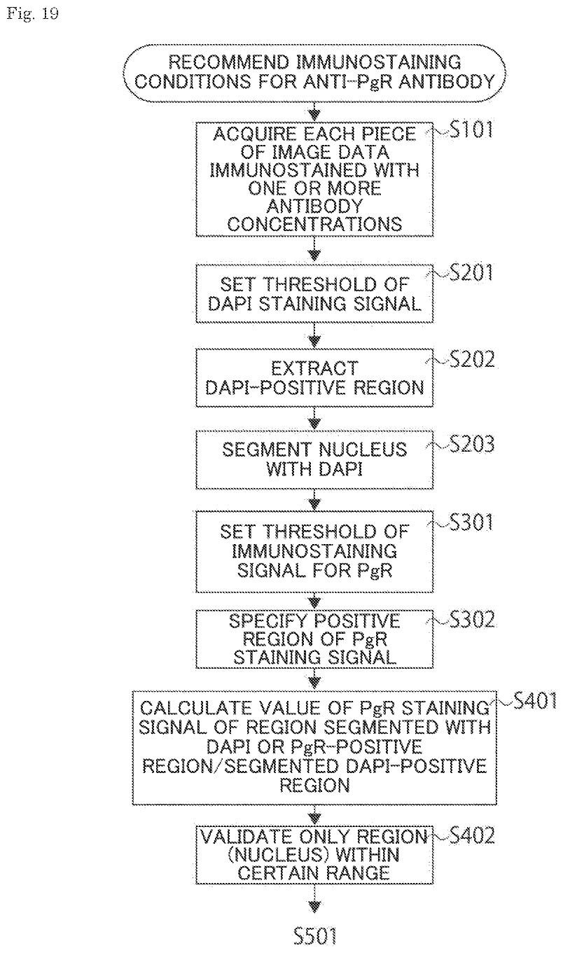

FIG. 19 is a flowchart showing a fourth embodiment of the method of presenting immunostaining conditions for anti-PgR antibodies using the information processing device according to the present technology.

Antibody conditions: conditions with one or more antibody concentrations

Specimen: when an image including positive cells and negative cells in one specimen is used.

Staining signal: value of fluorescence signal

Database reference: none

First, each piece of image data immunostained with one or more antibody concentrations is acquired (S101).

Next, in order to determine morphology information, the threshold value of the DAPI staining signal is set (S201), and the positive region of the DAPI staining signal is extracted (S202). In addition, nucleus segmentation is performed with DAPI to determine the morphology of nuclei (S203).

Next, the threshold value of the PgR staining signal is set (S301), and the positive region of the PgR staining signal is specified (S302).

The value of the PgR staining signal in the region segmented with DAPI or the percentage of the PgR-positive region in the DAPI region (PgR-positive region/segmented DAPI-positive region) for each segmented DAPI-positive region is calculated (S401).

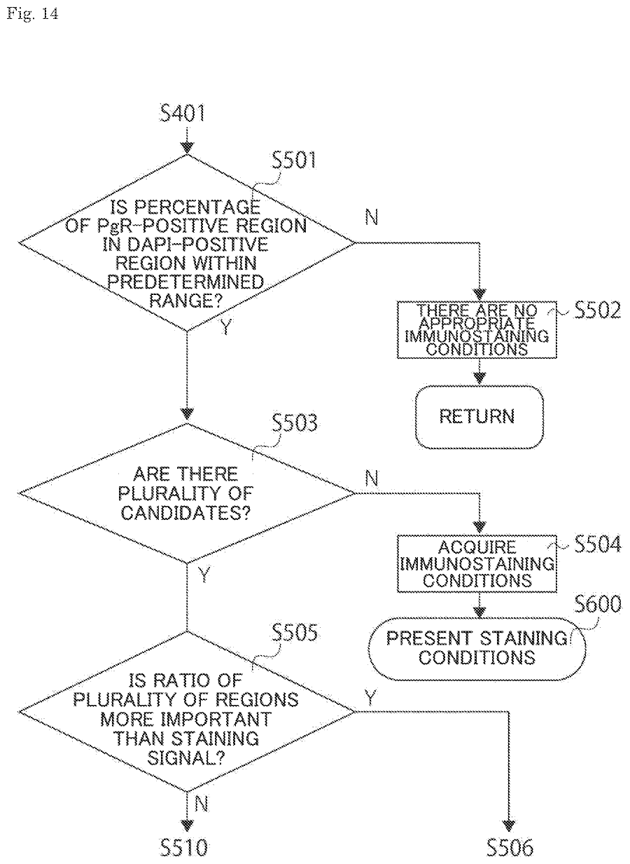

In this case, only the region (nucleus) within a certain range is validated (S402). The steps after this are the same as S501 to S604 of the first embodiment shown in FIG. 14 to FIG. 16.

### Fifth Embodiment

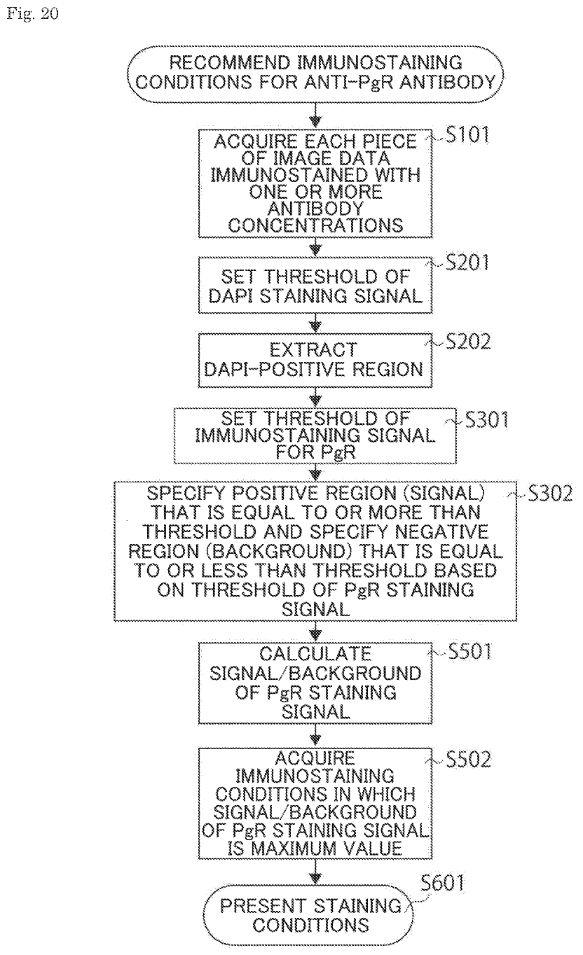

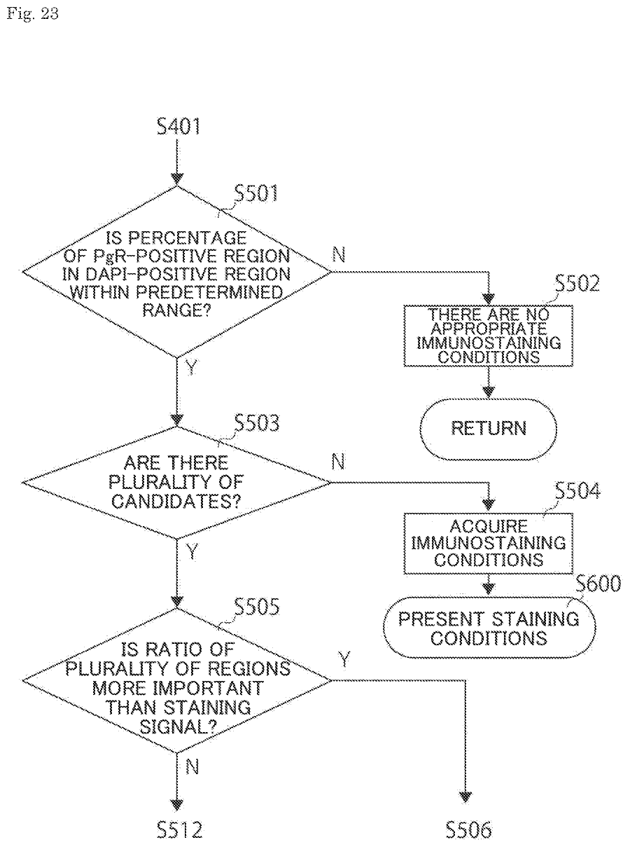

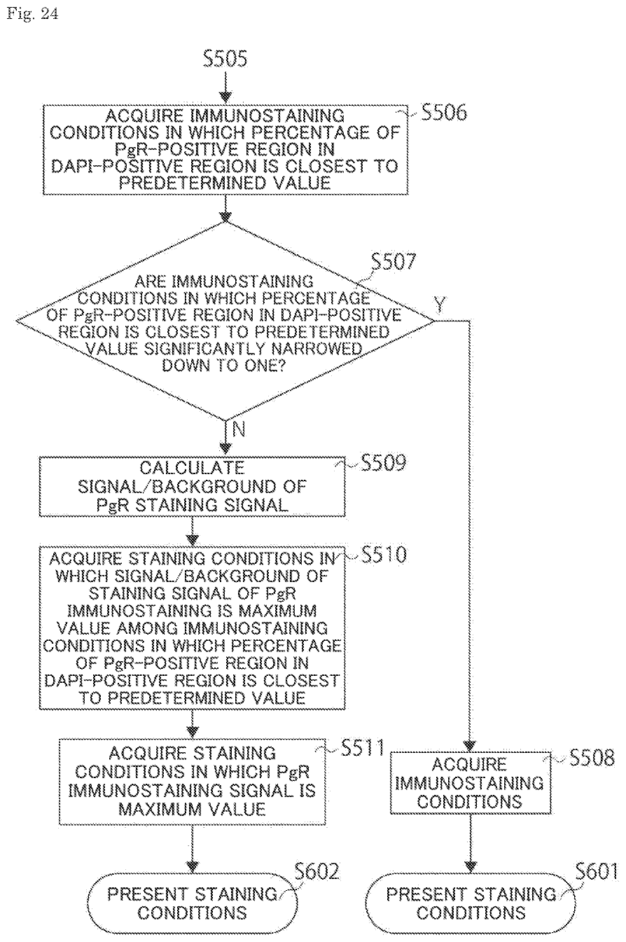

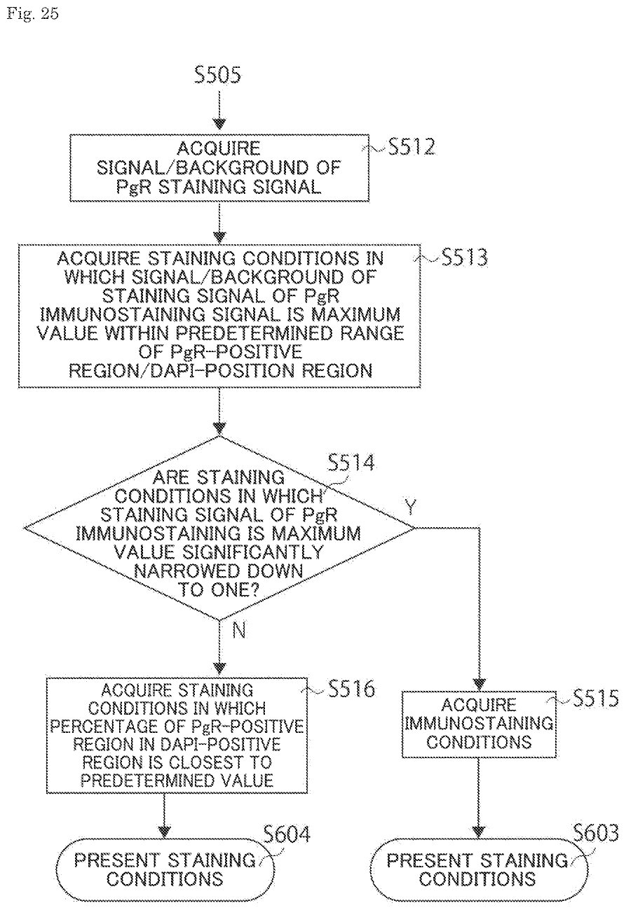

FIG. 20 to FIG. 25 are flowcharts showing a fifth embodiment of the method of presenting immunostaining conditions for anti-PgR antibodies using the information processing device according to the present technology.

Antibody conditions: conditions with one or more antibody concentrations

Specimen: when the specimen contains only positive cells

Staining signal: signal/background

Database reference: none

First, each piece of image data immunostained with one or more antibody concentrations is acquired (S101).

Next, the threshold value of the DAPI staining signal is set (S201), and the positive region of the DAPI staining signal is extracted (S202).

Next, the threshold value of the PgR staining signal is set (S301), and based on the set threshold value of the PgR staining signal, a positive region (signal) that is equal to or more than the threshold value and a negative region (background) that is equal to or less than the threshold value are specified (S302), and the signal/background of the PgR staining signal is calculated (S501).

### Fifth Embodiment-11: FIG. 20

Immunostaining conditions in which the calculated signal/background of the PgR staining signal is the maximum value are acquired (S502), and the acquired immunostaining conditions are presented (S601).

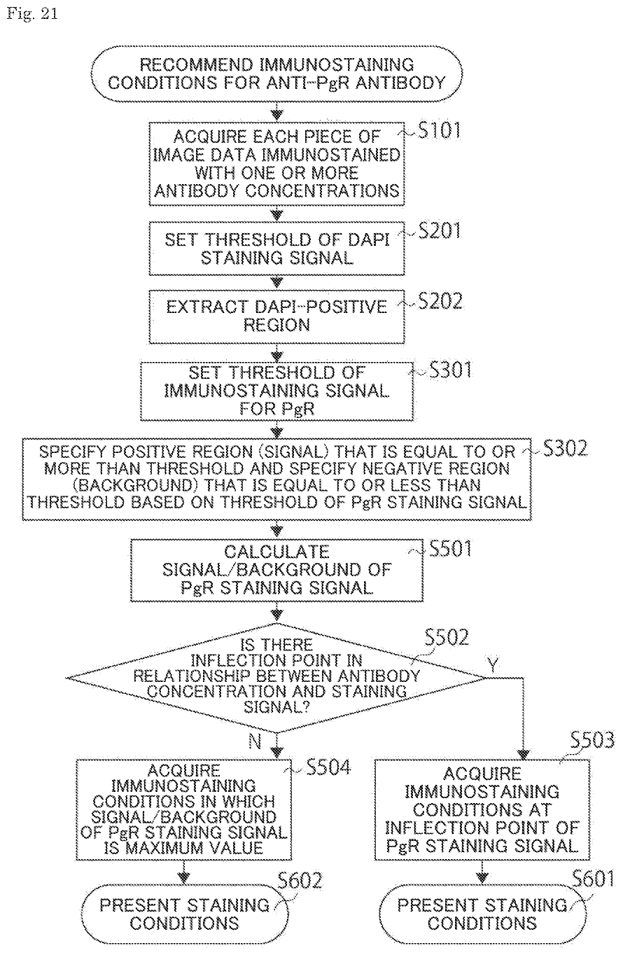

### Fifth Embodiment-21: FIG. 21

When there is an inflection point in the relationship between the antibody concentration and the staining signal (S502), immunostaining conditions at the inflection point are acquired (S503), and the acquired immunostaining conditions are presented (S601).

When there is no inflection point in the relationship between the antibody concentration and the staining signal (S502), immunostaining conditions in which the calculated signal/background of the PgR staining signal is the maximum value are acquired (S504), and the acquired immunostaining conditions are presented (S602).

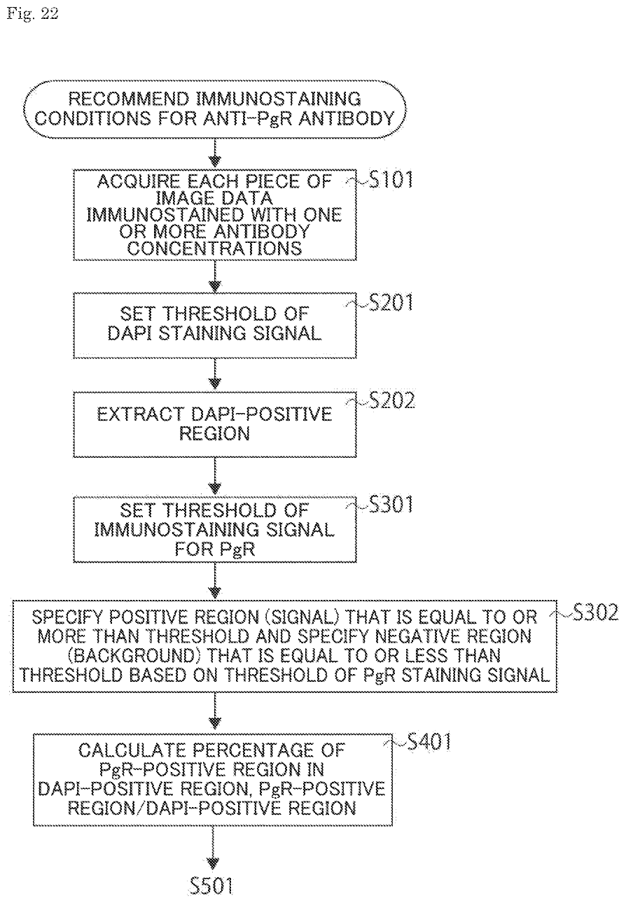

### Fifth Embodiment-31: FIG. 22 to FIG. 25

A percentage of the PgR-positive region in the DAPI-positive region (PgR-positive region/DAPI-positive region) is calculated (S401).

When the percentage of the PgR-positive region in the DAPI-positive region is not within a predetermined range (S501), it is determined that that there are no appropriate immunostaining conditions (S502), and the process returns, and when the percentage of the PgR-positive region in the DAPI-positive region is within a predetermined range (S501), and when there are no plural candidates (S503), immunostaining conditions are acquired (S504), and the acquired immunostaining conditions are presented (S600).

When the percentage of the PgR-positive region in the DAPI-positive region is within a predetermined range and there are a plurality of candidates (S503), and when the ratio of the plurality of regions is more important than the staining signal (S505), immunostaining conditions in which the percentage of the PgR-positive region in the DAPI-positive region is closest to the predetermined value are acquired (S506). When the acquired immunostaining conditions are significantly narrowed down to one (S507), immunostaining conditions are acquired (S508), and the acquired immunostaining conditions are presented (S601).

When the acquired immunostaining conditions are not significantly narrowed down to one (S507), the signal/background of the PgR staining signal is calculated (S509). Among immunostaining conditions in which the percentage of the PgR-positive region in the DAPI-positive region is closest to an arbitrary value, staining conditions in which the signal/background of the PgR staining signal is the maximum value are acquired (S510), and additionally, staining conditions in which the PgR staining signal is the maximum value are acquired (S511), and the acquired immunostaining conditions are presented (S602).

When the percentage of the PgR-positive region in the DAPI-positive region is within a predetermined range and there are a plurality of candidates (S503), and when the ratio of the plurality of regions is less important than the staining signal (S505), first, the signal/background of the PgR staining signal is calculated (S512). Within the predetermined range of PgR-positive region/DAPI-positive region, staining conditions in which the signal/background of the PgR staining signal is the maximum value are acquired (S513). When staining conditions in which the PgR immunostaining signal is the maximum value are significantly narrowed down to one (S514), immunostaining conditions are acquired (S515), and the acquired immunostaining conditions are presented (S603).

When staining conditions in which the PgR immunostaining signal is the maximum value are not significantly narrowed down to one (S514), immunostaining conditions in which the percentage of the PgR-positive region in the DAPI-positive region is closest to an arbitrary value are acquired (S516), and the acquired immunostaining conditions are presented (S604).

### Sixth Embodiment

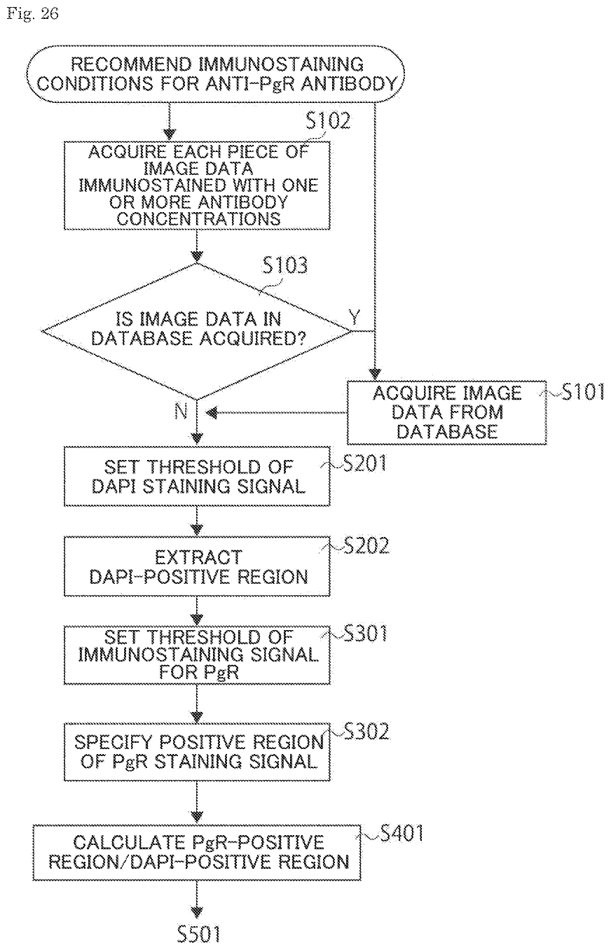

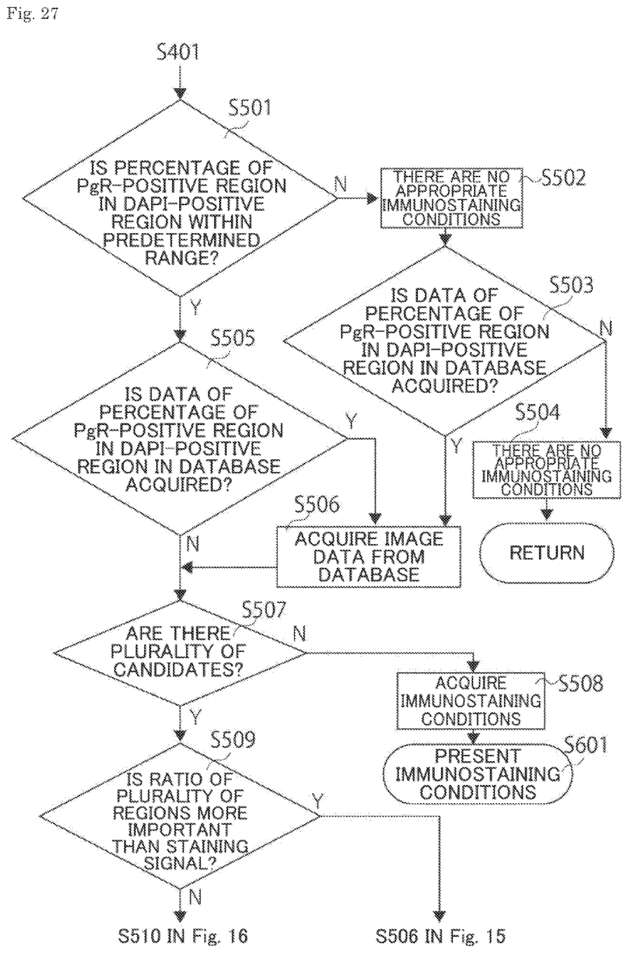

FIG. 26 and FIG. 27 are flowcharts showing a sixth embodiment of the method of presenting immunostaining conditions for anti-PgR antibodies using the information processing device according to the present technology.

Antibody conditions: conditions with one or more antibody concentrations

Specimen: when the specimen contains only positive cells

Staining signal: value of fluorescence signal

Database reference: yes

First, each piece of image data immunostained with one or more antibody concentrations is acquired (S102). When image data in the database is acquired (S103), the image data is acquired from the database (S101).

Next, the threshold value of the DAPI staining signal is set (S201), and the positive region of the DAPI staining signal is extracted (S202).

Next, the threshold value of the PgR staining signal is set (S301), and the positive region of the PgR staining signal is specified (S302).

A percentage of the PgR-positive region in the DAPI-positive region (PgR-positive region/DAPI-positive region) is calculated (S401).

When the percentage of the PgR-positive region in the DAPI-positive region is not within a predetermined range (S501), it is determined that that there are no appropriate immunostaining conditions (S502), and it is determined whether PgR-positive region/DAPI-positive region data in the database is acquired (S503). When the database is not referred to, it is determined that that there are no appropriate immunostaining conditions (S504), and the process returns.

On the other hand, when the percentage of the PgR-positive region in the DAPI-positive region is within a predetermined range and there are a plurality of candidates (S501), it is determined whether PgR-positive region/DAPI-positive region data in the database is acquired (S505).

When the database is not referred to, and regarding the PgR-positive region/DAPI-positive region calculated in S401, if the database is referred to, image data is acquired from the database (S506), and regarding the PgR-positive region/DAPI-positive region calculated in S401 and PgR-positive region/DAPI-positive region data in the database, when there are a plurality of candidates (S507), immunostaining conditions are acquired (S508), and the acquired immunostaining conditions are presented (S601).

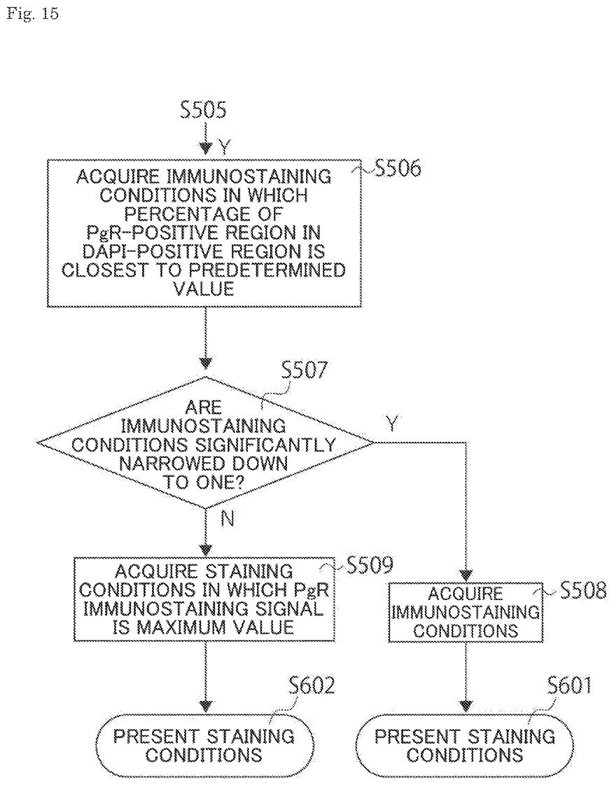

When there are a plurality of candidates (S507), and when the ratio of the plurality of regions is more important than the staining signal (S509), the same flow as S506 to S602 of the first embodiment shown in FIG. 15 is performed.

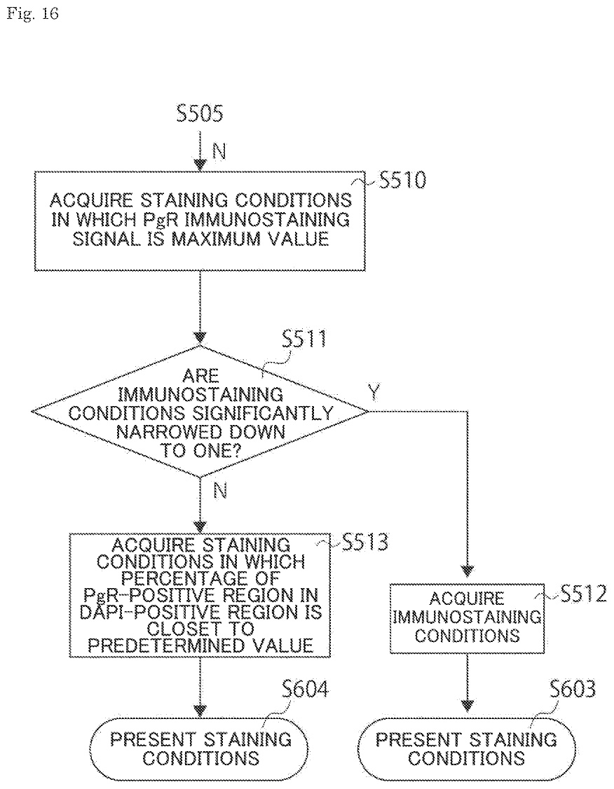

When there are a plurality of candidates (S507), and when the ratio of the plurality of regions is less important than the staining signal (S509), the same flow as S510 to S604 of the first embodiment shown in FIG. 16 is performed.

### Seventh Embodiment

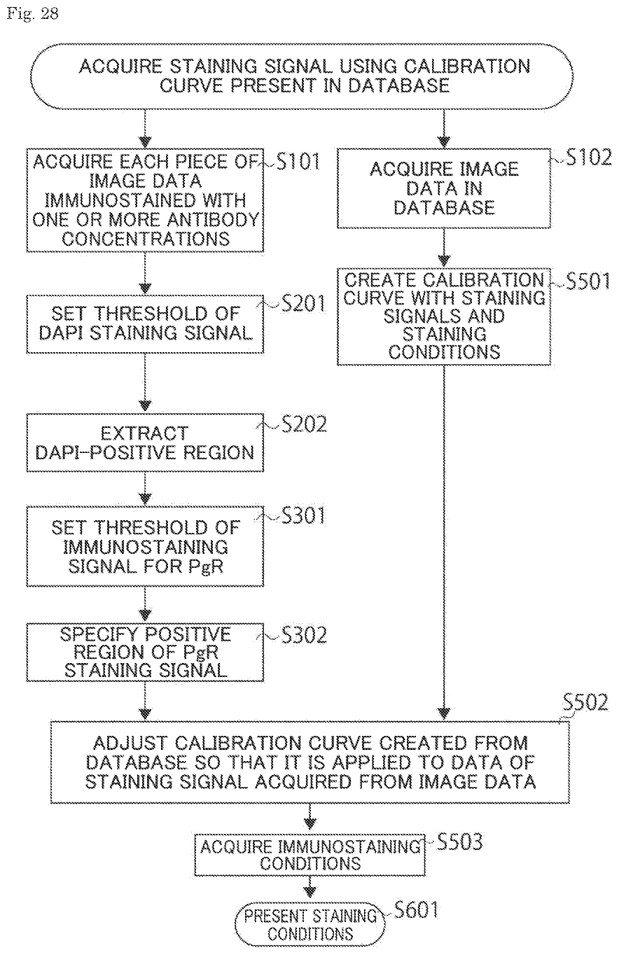

FIG. 28 is a flowchart showing a seventh embodiment of the method of presenting immunostaining conditions for anti-PgR antibodies using the information processing device according to the present technology.

Antibody conditions: conditions with one or more antibody concentrations

Specimen: when the specimen contains only positive cells

Staining signal: value of fluorescence signal

Database reference: yes

First, each piece of image data immunostained with one or more antibody concentrations is acquired (S101).

Next, like S201 to S302 of the first embodiment, staining signal data is acquired (S201 to S302).

On the other hand, image data in the database is acquired (S102), and a calibration curve is created with staining signals and staining conditions (S501). A calibration curve created from the database is adjusted so that it is applied to the staining signal data acquired in S101 to S302 (S502). Immunostaining conditions are acquired from the adjusted calibration curve (S503), and the acquired immunostaining conditions are presented (S601).

Here, in the present technology, the following configurations can be used.

(1) An information processing device, including:

a signal acquisition unit that acquires a signal derived from a sample including a biological sample;

a processing unit that calculates immunostaining conditions of a reagent for the sample based on the signal; and

an output unit that outputs the immunostaining conditions,

wherein the signal includes a signal derived from the reagent containing target molecules in the biological sample and binding molecules that are able to bind to the target molecules and/or a signal derived from non-target molecules and the reagent.

(2) The information processing device according to (1),

wherein the signal includes at least one of a signal, a specific signal/background, and a specific signal/non-specific signal.

(3) The information processing device according to (1) or (2),

wherein the processing unit calculates immunostaining conditions of the reagent for the sample including the biological sample based on signals derived from the sample stained with a plurality of reagent concentrations and a threshold value.

(4) The information processing device according to (3),

wherein the threshold value is the maximum signal among the signals derived from the sample stained with a plurality of reagent concentrations.

(5) The information processing device according to any one of (1) to (3),

wherein the processing unit calculates immunostaining conditions of the reagent for the sample including the biological sample based on the signal derived from the sample stained with at least one reagent concentration and reagent information referred to in a database.

(6) The information processing device according to any one of (1) to (5),

wherein the processing unit calculates immunostaining conditions of the reagent for the sample including the biological sample based on the signals derived from the sample stained with a plurality of reagent concentrations and a threshold value extracted from region information.

(7) The information processing device according to (6),

wherein the processing unit determines a region based on the signal and/or bright field image.

(8) The information processing device according to (7),

wherein the region includes morphology information of the biological sample.

(9) The information processing device according to (8),

wherein the morphology information includes a cell membrane and a nuclear distribution.

(10) The information processing device according to (8) or (9),

wherein the morphology information includes a cell morphology obtained by segmentation.

(11) The information processing device according to any one of (7) to (9),

wherein the processing unit compares the plurality of determined regions.

(12) The information processing device according to (11),

wherein the processing unit compares the plurality of regions including at least one of a cell membrane, a cell nucleus, a specific binding region and a non-specific binding region and analyzes localization of the regions.

(13) The information processing device according to (11) or (12),

wherein, in the processing unit, the plurality of determined regions are single cells, and

wherein the processing unit excludes signals derived from single cells that overlap and/or are adjacent to each other within a predetermined distance.

(14) The information processing device according to any one of (1) to (13),

wherein the signal acquisition unit acquires a fluorescence signal after autofluorescence separation and/or inter-dye color separation.

(15) The information processing device according to any one of (1) to (14),

wherein the output unit outputs, as the immunostaining conditions, at least one of an antibody clone, an antibody concentration, an antigen-antibody reaction time, a reaction temperature, antigen activation conditions, a composition of a reaction solution, and stirring conditions.

(16) The information processing device according to any one of (1) to (15), further including a presentation unit that presents support information for staining conditions to a user based on the output immunostaining conditions.

(17) An information processing method, including:

a signal acquisition step in which a signal derived from a sample including a biological sample is acquired;

a processing step in which immunostaining conditions of a reagent for the sample are calculated based on the signal; and

an output step in which the immunostaining conditions are output,

wherein the signal includes a signal derived from a reagent containing target molecules in the biological sample and binding molecules that are able to bind to the target molecules and/or a signal derived from non-target molecules and the reagent.

(18) A computer program causing a computer to implement:

a signal acquisition function of acquiring a signal derived from a sample including a biological sample;

a processing function of calculating immunostaining conditions of a reagent for the sample based on the signal; and

an output function of outputting the immunostaining conditions,

wherein the signal includes a signal derived from a reagent containing target molecules in the biological sample and binding molecules that are able to bind to the target molecules and/or a signal derived from non-target molecules and the reagent.

(19) A target molecule detection analysis system, including:

a signal acquisition unit that acquires a signal derived from a sample including a biological sample;

a processing unit that calculates immunostaining conditions of a reagent for the sample based on the signal;

an output unit that outputs the immunostaining conditions; and

a detection unit that detects a signal derived from the stained sample based on the output immunostaining condition information,

wherein the signal includes a signal derived from a reagent containing target molecules in the biological sample and binding molecules that are able to bind to the target molecules and/or a signal derived from non-target molecules and the reagent.

(20) The target molecule detection system according to (19), further including

a staining unit that stains the sample using the reagent.

(21) The target molecule detection system according to (19) or (20), further including

an analysis unit that analyzes the sample based on the signal.

### REFERENCE SIGNS LIST

1 Information processing device

11 Signal acquisition unit

12 Processing unit

13 Output unit

14 Presentation unit

15 Storage unit

16 Display unit

17 User interface

2 Information processing system

3 Target molecule detection device

4 Target molecule detection system

31 Detection unit

41 Detection device

32 Staining unit

42 Staining device

33 Analysis unit

43 Analysis device

1. An information processing device, comprising:
a signal acquisition unit that acquires a signal derived from a sample including a biological sample;
a processing unit that calculates immunostaining conditions of a reagent for the sample based on the signal; and
an output unit that outputs the immunostaining conditions,
wherein the signal includes a signal derived from the reagent containing target molecules in the biological sample and binding molecules that are able to bind to the target molecules and/or a signal derived from non-target molecules and the reagent.

2. The information processing device according to claim 1,
wherein the signal includes at least one of a signal, a specific signal/background, and a specific signal/non-specific signal.

3. The information processing device according to claim 1,
wherein the processing unit calculates immunostaining conditions of the reagent for the sample including the biological sample based on signals derived from the sample stained with a plurality of reagent concentrations and a threshold value.

4. The information processing device according to claim 3,
wherein the threshold value is the maximum signal among the signals derived from the sample stained with a plurality of reagent concentrations.

5. The information processing device according to claim 1,
wherein the processing unit calculates immunostaining conditions of the reagent for the sample including the biological sample based on the signal derived from the sample stained with at least one reagent concentration and reagent information referred to in a database.

6. The information processing device according to claim 1,
wherein the processing unit calculates immunostaining conditions of the reagent for the sample including the biological sample based on the signals derived from the sample stained with a plurality of reagent concentrations and a threshold value extracted from region information.

7. The information processing device according to claim 6,
wherein the processing unit determines a region based on the signal and/or bright field image.

8. The information processing device according to claim 7,
wherein the region includes morphology information of the biological sample.

9. The information processing device according to claim 8,
wherein the morphology information includes a cell membrane and a nuclear distribution.

10. The information processing device according to claim 8,
wherein the morphology information includes a cell morphology obtained by segmentation.

11. The information processing device according to claim 7,
wherein the processing unit compares the plurality of determined regions.

12. The information processing device according to claim 11,
wherein the processing unit compares the plurality of regions including at least one of a cell membrane, a cell nucleus, a specific binding region and a non-specific binding region and analyzes localization of the regions.

13. The information processing device according to claim 11,
wherein the plurality of determined regions are single cells, and
wherein the processing unit excludes signals derived from single cells that overlap and/or are adjacent to each other within a predetermined distance.

14. The information processing device according to claim 1,
wherein the signal acquisition unit acquires a fluorescence signal after autofluorescence separation and/or inter-dye color separation.

15. The information processing device according to claim 1,
wherein the output unit outputs, as the immunostaining conditions, at least one of an antibody clone, an antibody concentration, an antigen-antibody reaction time, a reaction temperature, antigen activation conditions, a composition of a reaction solution, and stirring conditions.

16. The information processing device according to claim 1, further comprising
a presentation unit that presents support information for staining conditions to a user based on the output immunostaining conditions.

17. An information processing method, comprising:
a signal acquisition step in which a signal derived from a sample including a biological sample is acquired;
a processing step in which immunostaining conditions of a reagent for the sample are calculated based on the signal; and
an output step in which the immunostaining conditions are output,
wherein the signal includes a signal derived from a reagent containing target molecules in the biological sample and binding molecules that are able to bind to the target molecules and/or a signal derived from non-target molecules and the reagent.

18. A computer program causing a computer to implement:
a signal acquisition function of acquiring a signal derived from a sample including a biological sample;
a processing function of calculating immunostaining conditions of a reagent for the sample based on the signal; and
an output function of outputting the immunostaining conditions,
wherein the signal includes a signal derived from a reagent containing target molecules in the biological sample and binding molecules that are able to bind to the target molecules and/or a signal derived from non-target molecules and the reagent.

19. A target molecule detection system, comprising:
a signal acquisition unit that acquires a signal derived from a sample including a biological sample;
a processing unit that calculates immunostaining conditions of a reagent for the sample based on the signal;
an output unit that outputs the immunostaining conditions; and
a detection unit that detects a signal derived from the stained sample based on the output immunostaining conditions,
wherein the signal includes a signal derived from a reagent containing target molecules in the biological sample and binding molecules that are able to bind to the target molecules and/or a signal derived from non-target molecules and the reagent.

20. The target molecule detection system according to claim 19, further comprising
a staining unit that stains the sample using the reagent.

21. The target molecule detection system according to claim 19, further comprising
an analysis unit that analyzes the sample based on the signal detected by the detection unit.
